(19) United States
(12) Patent Application Publication
(10) Pub. No.: US 2018/0303757 A1
(43) Pub. Date: Oct. 25, 2018

(54) ENHANCED ABUSE-DETERRENT FORMULATIONS OF OXYCODONE

(71) Applicant: KASHIV PHARMA LLC, Bridgewater, NJ (US)

(72) Inventors: Siddhartha Banerjee, West Windsor, NJ (US); Navnit H. Shah, Clifton, NJ (US); Gaurang Patel, Iselin, NJ (US); Murali Mohan Bommana, Bridgewater, NJ (US); Ashish Chatterji, East Brunswick, NJ (US); Anita Kumar, Miller Place, NY (US); Petra Inbar, Glen Rock, NJ (US); Furong Liu, Basking Ridge, NJ (US); Srinivas Kone, Monroe, NJ (US); Priti L. Jagani, Hillsborough, NJ (US); Wantanee Phuapradit, Montville, NJ (US)

(73) Assignee: KASHIV PHARMA LLC, Bridgewater, NJ (US)

(21) Appl. No.: 15/770,021

(22) PCT Filed: Oct. 21, 2016
(86) PCT No.: PCT/US2016/058256

§ 371 Date: Apr. 20, 2018

Related U.S. Application Data
(60) Provisional application No. 62/245,651, filed on Oct. 23, 2015.

(51) Int. Cl. A61K 9/20; A61K 31/485

(52) U.S. Cl. CPC A61K 9/2031; A61K 31/485; A61K 9/2013; A61K 9/2009; A61K 9/2054; A61K 9/2027; A61K 9/2095

(57) ABSTRACT

The present technology relates to an extended-release solid oral pharmaceutical composition, comprising a cured blend of a melt-extruded first component and a second component, wherein the melt-extruded first component comprises a therapeutically effective amount of an opioid or a pharmaceutically acceptable salt thereof, at least one PEO polymer, and a stabilizing agent; and the second component comprises at least one PEO polymer and an oxidative stabilizing agent. The extended-release pharmaceutical compositions of the present technology provide crush-resistant and abuse-deterrent formulations featuring enhanced heat stability, resistance to drug segregation, and resistance to alcohol-induced dose dumping.

This application claims priority to U.S. Provisional Patent Application No. 62/245,651, filed Oct. 23, 2015, the disclosure of which is incorporated by reference herein in its entirety.

### TECHNICAL FIELD

The present invention relates generally to the field of extended release opioid pharmaceutical formulations exhibiting improved resistance to tampering and abuse. More specifically, for example, the present invention relates to extended release opioid pharmaceutical formulations with enhanced heat stability and resistance to drug segregation, both of which contribute to the improved resistance to tampering and abuse.

### BACKGROUND

Pharmaceutical products are sometimes the subject of abuse. For example, a particular dose of an opioid may be more potent when administered parenterally as compared to the same dose administered orally. Some opioid pharmaceutical formulations, including extended release/controlled release opioid pharmaceutical formulations, can be tampered with to provide the opioid contained therein for illicit use.

Methods for abusing prescription pharmaceutical compositions are varied and include, but are not limited to, extraction, melting, volatilization, physical tampering (e.g., grinding, grating, crushing, etc.), or direct administration. For purposes of abuse, methods of administering active drug substances obtained from prescription pharmaceutical compositions, or the pharmaceutical compositions themselves, are similarly diverse and include, for example, injection, smoking, snorting, swallowing, sublingual or buccal administration, chewing, and administration as a suppository. Alcohol-induced dose dumping of active drug substance from prescription pharmaceutical compositions also presents potential abuse and safety problems. As a result of the continued encouragement by the U.S. Food and Drug Administration (FDA) to develop abuse-deterrent formulations, there have been a number of attempts in the art to control the abuse potential associated with opioid drugs.

For example, U.S. Pat. Nos. 8,894,987 and 8,894,988 disclose an abuse-deterrent dosage form that is crush resistant and resistant to alcohol extraction. The '987 and '988 patents are directed to a pharmaceutical composition created by (a) combining (1) at least one polyethylene oxide (“PEO”) polymer having, based on rheological measurements, an approximate molecular weight of at least 1,000,000; and (2) at least one active agent; (b) shaping the composition to form an extended release matrix formulation; and (c) curing the extended release matrix formulation comprising at least a curing step of subjecting the extended release matrix formulation to a temperature which is at least the melting temperature of PEO for a time period of at least about 1 minute.

These compositions claim to (1) be crush resistant, (2) be alcohol resistant, (3) be resistant to methods of extraction, and (4) prevent syringeability, the latter by rapidly forming a gelatinous mass that resists passage through a needle when subjected to aqueous media.

U.S. Pat. No. 8,808,741 is directed to convection-cured, extended release oxycodone tablets that are prepared by combining PEO and oxycodone to form a blend, shaping the blend into a tablet, and convection curing the tablet.

U.S. Pat. Nos. 8,309,060 and 8,114,383 describe a thermoformed dosage form that contains one or more abuse-prone active agents, one synthetic or natural polymer, and optionally a wax. The dosage form exhibits a breaking strength of at least 500 N.

U.S. Pat. No. 8,337,888 describes a controlled-release oral dosage form containing oxycodone and a gelling agent to impart a viscosity of at least about 10 cP when dissolved in 0.5 ml to about 10 ml of an aqueous liquid. Other documents, such as U.S. Pat. No. 7,776,314, describe the inclusion of a viscosity-increasing agent in a dosage form of an abuse-prone active agent such that an aqueous extract from the dosage form produces a gel that cannot pass through a needle.

U.S. Pat. No. 8,840,928 describes tamper-resistant pharmaceutical compositions comprising a plurality of solid particles, each particle comprising a solid solution comprising: (a) one or more drugs prone to abuse; and (b) one or more fatty acids; wherein the one or more drugs interact ionically with the one or more fatty acids; and the one or more fatty acids comprise at least about 42%-69% by weight of the particle.

OXYCONTIN® reformulated tablets (ORT; ORTs; ORT tablets) are currently marketed as a controlled-release abuse-deterrent oxycodone formulation. ORTs purportedly are more difficult to crush, break, or dissolve in an aqueous or alcoholic solution, and also form a viscous hydrogel that cannot easily be injected. However, the present applicants have found that ORTs can still be abused by chewing or by ease of extractability from the ground tablets. Results with the extended release/controlled release ORT 40 mg formulation demonstrated that the abuse-deterrent properties with regard to alcohol-induced dose dumping protection, syringeability protection, and extractability protection are defeated by heat pretreatment, crushing/cutting, or heat pretreatment followed by crushing/cutting. Furthermore, the oxycodone in ORT formulations has a tendency to segregate out as a free drug from the extended release matrix when subjected to harsh grinding (e.g., by coffee grinder). This free drug can be easily snorted by an abuser, or can be easily extracted into water and subsequently injected by an abuser.

ORT tablets include a small amount of butylated hydroxyl toluene (BHT), which is present as an antioxidant in, for example, PEO polymer. Possible methods to defend a dosage form from an oxidative degradation process include addition of antioxidants, storage under an inert atmosphere, or application of an oxygen barrier film coating. The latter two methods are, however, difficult to apply during all stages of the manufacturing process. It is further known that oxidative degradation processes are especially accelerated when the dosage forms are exposed to harsh processing conditions, for example, during the manufacturing process. For example, high molecular weight PEO tends to degrade upon hot melt extrusion or melt granulation. Polymer degradation may result in an uncontrolled release profile, particularly when active material is embedded in a matrix of PEO; this might contribute to oxidative degradation of pharmacologically active ingredients by radicals. When adding a potentially suitable excipient, such as BHT, in order to stabilize high molecular weight PEO polymer, it should be taken into consideration that such an excipient may be unstable or volatile at elevated temperatures, such as those employed during hot melt extrusion or melt granulation in the manufacture of ORTs, or used by an abuser to defeat the extended release property.

Accordingly, there remains a need for extended release, abuse-deterrent formulations of abuse-prone drugs that can maintain an extended release/controlled release profile even after being subjected to methods of abuse, such as heating (e.g., heat pretreatment) and/or crushing/grinding.

### SUMMARY

In some embodiments, there are provided abuse-deterrent oral tablets providing an extended release of an active agent, such as an opioid.

In some embodiments, there are provided methods of deterring abuse of an opioid, comprising providing an abuse-deterrent tablet to a subject in need thereof.

In some embodiments, there are provided methods of decreasing the abuse potential of an opioid, comprising providing an abuse-deterrent oral tablet to a subject in need thereof.

In some embodiments, there are provided methods for treating or preventing pain in a subject in need thereof, comprising administering a therapeutically effective amount of an opioid to the subject, wherein the opioid is in an abuse-deterrent oral tablet dosage form.

Certain embodiments include an oral tablet composition comprising a first component and a second component, wherein the first component comprises a therapeutically effective amount of oxycodone or a pharmaceutically acceptable salt thereof at least one polyethylene oxide (PEO) polymer; a stabilizing agent; and, optionally, at least one rate limiting polymer; and the second component comprises at least one PEO polymer; and a stabilizing agent, and wherein the first component is hot melt extruded or melt granulated, milled, and blended with the second component, and compressed into a tablet, and the tablet is cured. In an embodiment of the oral tablet composition, the tablet provides extended release/controlled release of oxycodone and exhibits enhanced heat stability and higher resistance to drug segregation compared to an extended release oxycodone oral tablet that is not made by a combination of hot melt extrusion (or melt granulation) and curing. In another embodiment of the oral tablet composition, the PEO polymers in the first component and in the second component independently have a weight average molecular weight of about 900,000 Dalton (Da) to about 7,000,000 Da. In an embodiment of the oral tablet composition, the PEO polymers in the first component and in the second component have different weight average molecular weights. For example, in an embodiment of the oral tablet composition, the PEO polymer in the first component has a weight average molecular weight of less than 1,000,000 Da (i.e., a low molecular weight PEO polymer) and the PEO polymer in the second component has a weight average molecular weight of at least (i.e., greater than or equal to) 1,000,000 Da (i.e., a high molecular weight PEO polymer). In some embodiments of the oral tablet composition, the tablet comprises less than about 35% by weight (based on the total weight of the composition) of the PEO polymer with a weight average molecular weight of at least 1,000,000 Da. In some embodiments of the oral tablet composition, the tablet comprises less than about 65% by weight (based on the total weight of the composition) of the total combined weight of high and low molecular weight PEO polymers. In some embodiments of the oral tablet composition, the PEO polymers in the first component and in the second component have the same weight average molecular weights. In some embodiments of the oral tablet composition, the at least one rate limiting polymer is a nonionic pH-independent polymer selected from the group consisting of hydroxypropyl methyl cellulose (HPMC); hydroxypropyl cellulose (HPC); polyvinyl pyrrolidone (PVP); polyvinyl acetate (PVA); cellulose acetate (CA); a polymer comprising a mixture of PVA, PVP, sodium lauryl sulfate, and silica; and mixtures thereof. For example, in an embodiment, the at least one rate limiting polymer comprises (1) HPMC and (2) a polymer comprising a mixture of PVA, PVP, sodium lauryl sulfate, and silica. In some embodiments of the oral tablet composition, the stabilizing agent is an antioxidant. For example in certain embodiments, the antioxidant provides heat stability (e.g., enhanced heat stability) to the PEO polymer during the hot melt extrusion or melt granulation process, and/or the antioxidant provides heat stability to the PEO polymer during the curing process, and/or the antioxidant prevents oxidative degradation of oxycodone or a pharmaceutically acceptable salt thereof, and/or the antioxidant prevents auto-oxidation of the PEO polymer. In some embodiments of the oral tablet composition, the antioxidant is selected from the group consisting of d-alpha-tocopherol, polyethylene glycol 1000 succinate, ascorbic acid, dl-alpha-tocopherol, dl-alpha-tocopherol acetate, alpha-tocopherol, vitamin E, sodium citrate, and citric acid. In some embodiments, the antioxidant is dl-alpha-tocopherol. In some embodiments of the oral tablet composition, the tablet is cured by heating at a temperature of between about 65° C. and about 100° C. For example, in certain embodiments, the tablet is cured by heating at a temperature of about 75° C. In some embodiments of the oral tablet composition, the tablet is cured for a period of about 11 hours to about 24 hours. For example, in certain embodiments, the tablet is cured for a period of about 16 hours. In some embodiments of the oral tablet composition, the tablet provides a therapeutic plasma level of oxycodone for about 6 to about 24 hours. For example, in certain embodiments, the tablet provides a therapeutic plasma level of oxycodone for about 8 to about 24 hours. In certain embodiments, the tablet provides a therapeutic plasma level of oxycodone for about 12 to about 24 hours. In some embodiments of the oral tablet composition, the first component further comprises a plasticizer, a disintegrant, a surfactant, a wax, or a mixture thereof. In some embodiments of the oral tablet composition, the composition further comprises additional abuse deterrents selected from the group consisting of bittering agents, irritants, and dyes. In some embodiments of the oral tablet composition, the composition further comprises an opioid antagonist. For example, in certain embodiments, the opioid antagonist is selected from the group consisting of naltrexone, 6-β naltrexol, nalbuphine, nalmefene, naloxone, cyclazocine, levallorphan, cyclorphan, and oxilorphan. In certain embodiments, the opioid antagonist is naloxone. In some embodiments of the oral tablet composition, the tablet maintains an extended release profile of oxycodone after being subjected to heating (e.g., heat pretreatment) in an oven at a temperature of 100° C. for two hours, comparable to, or with a deviation of no more than about 20% (e.g., no more than about 15%, or about 10%, or about 5%, or intermediate values thereof) from, an extended release oxycodone oral tablet that is not subjected to heat pretreatment. In some embodiments of the oral tablet composition, the tablet maintains an extended release profile of oxycodone after being subjected to microwave radiation at 1200 W for about 14 minutes, comparable to, or with a deviation of no more than about 20% (e.g., no more than about 15%, or about 10%, or about 5%, or intermediate values thereof) from, an extended release oxycodone oral tablet that is not subjected to microwave radiation. In some embodiments of the oral tablet composition, the in vitro dissolution rate of the composition, characterized by the percentage of active released at 30 minutes of dissolution, when measured in a USP Apparatus I (basket) at 100 rpm in 900 ml simulated gastric fluid comprising 40% v/v ethanol at 37° C., after being subjected to heat pretreatment in an oven at a temperature of about 100° C. for about two hours, is comparable to, or deviates no more than about 20% (e.g., no more than about 15%, or about 10%, or about 5%, or intermediate values thereof) from, the in vitro dissolution rate of an extended release oxycodone oral tablet that is not subjected to heat pretreatment. In some embodiments of the oral tablet composition, the in vitro dissolution rate of the composition, characterized by the percentage of active released at 30 minutes of dissolution, when measured in a USP Apparatus I (basket) at 100 rpm in 900 ml simulated gastric fluid comprising 40% v/v ethanol at 37° C., after being subjected to heat pretreatment in an oven at a temperature of about 100° C. for about two hours, is comparable to, or deviates no more than about 20% (e.g., no more than about 15%, or about 10%, or about 5%, or intermediate values thereof) from, the corresponding in vitro dissolution rate, measured in a USP Apparatus I (basket) at 100 rpm in 900 ml simulated gastric fluid at 37° C. without ethanol. In some embodiments of the oral tablet composition, the in vitro dissolution rate of the composition, characterized by the percentage of active released at 30 minutes of dissolution, when measured in a USP Apparatus I (basket) at 100 rpm in 900 ml simulated gastric fluid comprising 40% ethanol at 37° C., deviates no more than 20% (e.g., no more than about 15%, or about 10%, or about 5%, or intermediate values thereof) from the corresponding in vitro dissolution rate, measured in a USP Apparatus I (basket) at 100 rpm in 900 ml simulated gastric fluid at 37° C., of the reference tablet (ORT). In some embodiments of the oral tablet composition, the in vitro dissolution rate of the composition, comprising the tablet cut into four pieces, characterized by the percent amount of active released at 30 minutes of dissolution when measured in a USP Apparatus I (basket) at 100 rpm in 900 ml simulated gastric fluid comprising 50% ethanol v/v at 37° C., after being subjected to heat pretreatment in an oven at a temperature of about 100° C. for about two hours, is comparable to, or deviates no more than 20% (e.g., no more than about 15%, or about 10%, or about 5%, or intermediate values thereof) from, the in vitro dissolution rate of an extended release oxycodone oral tablet that is not subjected to cutting and heat pretreatment. In some embodiments of the oral tablet composition, the oxycodone is fully embedded in a plasticized polymer matrix comprising PEO, HPMC, and a mixture of PVA, PVP, sodium lauryl sulfate, and silica. In some embodiments of the oral tablet composition, the composition is resistant to drug segregation from the matrix upon grinding. In some embodiments of the oral tablet composition, the resistance to drug segregation results in the percentage of oxycodone or a pharmaceutically acceptable salt thereof in the fines fraction of the ground tablet being close to that predicted from the composition of the tablet. For example, in certain embodiments, the resistance of drug segregation of oxycodone or a pharmaceutically acceptable salt thereof in the fines fraction upon grinding results in a reduced amount of oxycodone or a pharmaceutically acceptable salt thereof available for insufflation. In some embodiments of the oral tablet composition, the tablet provides an extended release of oxycodone and exhibits enhanced heat stability and higher resistance to drug segregation compared to another extended release oxycodone oral tablet.

Other embodiments of the present disclosure include a method of deterring abuse of oxycodone, the method comprising providing an oral tablet composition to a subject in need thereof, wherein the tablet comprises a first component and a second component, wherein the first component comprises a therapeutically effective amount of oxycodone or a pharmaceutically acceptable salt thereof; at least one PEO polymer; optionally, at least one rate limiting polymer; and a stabilizing agent; and the second component comprises at least one PEO polymer; and a stabilizing agent, and wherein the first component is hot melt extruded or melt granulated, milled, and blended with the second component, the blended components are compressed into a tablet, and the tablet is cured. In some embodiments of the method, the tablet provides an extended release of oxycodone and exhibits enhanced heat stability and higher resistance to drug segregation compared to an extended release oxycodone oral tablet that is not made by a combination of hot melt extrusion/melt granulation and curing. In some embodiments of the method, the stabilizing agent is dl-alpha-tocopherol. In some embodiments of the method, the tablet is cured for a period of about 16 hours.

Other embodiments of the present disclosure include a method of decreasing abuse potential of oxycodone, the method comprising providing an oral tablet composition to a subject in need thereof, wherein the tablet comprises a first component and a second component; wherein the first component comprises a therapeutically effective amount of oxycodone or a pharmaceutically acceptable salt thereof; at least one PEO polymer; optionally, at least one rate limiting polymer; and a stabilizing agent, and the second component comprises at least one PEO polymer and a stabilizing agent; and wherein the first component is hot melt extruded or melt granulated, milled, and blended with the second component, the blended components are compressed into a tablet, and the tablet is cured. In some embodiments of the method, the tablet provides an extended release of oxycodone and exhibits enhanced heat stability and higher resistance to drug segregation compared to an extended release oxycodone oral tablet that is not made by a combination of hot melt extrusion/melt granulation and curing. In some embodiments of the method, the stabilizing agent is dl-α-tocopherol. In some embodiments of the method, the tablet is cured for a period of about 16 hours.

Other embodiments of the present disclosure include a process for making an extended release, tamper resistant, abuse-deterrent oral tablet composition, comprising: mixing a therapeutically effective amount of oxycodone or a pharmaceutically acceptable salt thereof; at least one PEO polymer; optionally, at least one rate limiting polymer; and a stabilizing agent, and hot melt extruding or melt granulating the mixture to form a first component; milling the first component; mixing at least one PEO polymer and a stabilizing agent to form a second component; blending the first component and the second component to form a blended composition; compressing the blended composition to form a tablet; and curing the tablet. In some embodiments of the process, the tablet is cured by heating at a temperature of between 65° C. and 100° C. In some embodiments of the process, the tablet is cured by heating at a temperature of at least 75° C. In some embodiments of the process, the tablet is cured for a period of 11-24 hours. In some embodiments of the process, the tablet is cured by heating at a temperature of 75° C. for 16 hours. In some embodiments of the process, the stabilizing agent is dl-alpha-tocopherol.

Other embodiments of the present disclosure include an extended release, tamper-resistant, abuse-deterrent tablet composition comprising a first component and a second component, wherein the first component comprises a therapeutically effective amount of oxycodone or a pharmaceutically acceptable salt thereof; an antioxidant stabilizing agent; a PEO polymer; and at least one additional nonionic pH-independent polymer selected from the group consisting of HPMC and a PVA-based polymer; and the second component comprises a PEO polymer and an antioxidant stabilizing agent, and wherein the first component is hot melt extruded, milled, and blended with the second component, the blended components are compressed into a tablet, and the tablet is cured for at least 11 hours.

Yet further embodiments of the present disclosure include an extended release, tamper-resistant, abuse-deterrent tablet composition suitable for once or twice daily administration comprising a therapeutically effective amount of oxycodone or a pharmaceutically acceptable salt thereof; at least one PEO polymer; a stabilizing agent; and, optionally, at least one rate limiting polymer; wherein an in vitro dissolution profile of the dosage form, characterized by the percent amount of active released at 30 minutes of dissolution, when measured in a USP Apparatus I (basket) at 100 rpm in 900 ml simulated gastric fluid comprising 40% ethanol at 37° C., is comparable to, or deviates no more than 20% (e.g., no more than about 15%, or about 10%, or about 5%, or intermediate values thereof) from, the corresponding in vitro dissolution measured in a USP Apparatus I (basket) at 100 rpm in 900 ml simulated gastric fluid at 37° C. without ethanol, and wherein the tablet maintains the dissolution profiles after heat pretreatment in an oven at about 100° C. for about 2 hours, or after heat pretreatment by microwave irradiation at about 1200 W for about 14 minutes.

Other embodiments of the present disclosure include an extended release, tamper-resistant, abuse-deterrent tablet composition suitable for once or twice daily administration comprising a therapeutically effective amount of oxycodone or a pharmaceutically acceptable salt thereof; at least one PEO polymer; a stabilizing agent; and, optionally, at least one rate limiting polymer, wherein the composition is resistant to drug segregation upon grinding, and wherein the resistance to drug segregation results in the percentage of oxycodone or a pharmaceutically acceptable salt thereof in the fines fraction of the ground tablet being close to that predicted from the composition of the tablet.

Other embodiments of the present disclosure include an oral tablet composition comprising a first component and a second component, wherein the first component comprises oxycodone or a pharmaceutically acceptable salt thereof; at least one low molecular weight PEO polymer; at least one additional nonionic pH-independent polymer; and a stabilizing agent; and the second component comprises at least one high molecular weight PEO polymer; and a stabilizing agent, wherein the tablet comprises a dosage form having less than about 65% by weight, based on the total weight of the composition, of the total combined weight of high and low molecular weight PEO polymers, wherein the tablet comprises a dosage form having less than about 35% by weight, based on the total weight of the composition, of the high molecular weight PEO polymer, and wherein the first component is hot melt extruded or melt granulated, milled, and blended with the second component, and compressed into a tablet, and the tablet is cured.

In some embodiments, the present disclosure includes an extended release oral tablet composition comprising a matrix comprising a therapeutically effective amount of an opioid or a pharmaceutically acceptable salt thereof in combination with at least one low molecular weight polyethylene oxide (PEO) polymer having an approximate molecular weight less than 1,000,000 Dalton (Da) and, optionally, further comprising at least one high molecular weight PEO polymer having an approximate molecular weight of at least 1,000,000 Da; wherein the composition is hot melt extruded or melt granulated, and cured, and wherein the composition exhibits enhanced heat stability with a deviation of no more than a 20%, or a 15%, or a 10%, or a 5% increase in an in vitro dissolution rate in simulated gastric fluid (SGF) after heat pretreatment, when compared with corresponding in vitro dissolution rate in SGF without heat pretreatment. In some embodiments, the opioid or pharmaceutically acceptable salt thereof is oxycodone hydrochloride.

In some embodiments, the present disclosure includes an extended release oral tablet composition comprising a matrix comprising a therapeutically effective amount of an opioid or a pharmaceutically acceptable salt thereof in combination with at least one low molecular weight PEO polymer having an approximate molecular weight of less than 1,000,000 Da and, optionally, further comprising at least one high molecular weight PEO polymer having an approximate molecular weight of at least 1,000,000 Da; wherein the composition is hot melt extruded or melt granulated, and cured, and wherein the composition exhibits enhanced heat stability with a deviation of no more than a 20%, or a 15%, or a 10%, or a 5% increase in an in vitro dissolution rate in SGF containing 40% ethanol v/v after heat pretreatment, when compared with corresponding in vitro dissolution rate in SGF containing 40% ethanol v/v without heat pretreatment. In some embodiments, the opioid or pharmaceutically acceptable salt thereof is oxycodone hydrochloride.

In some embodiments, the present disclosure includes an extended release oral tablet composition comprising a matrix comprising a therapeutically effective amount of an opioid or a pharmaceutically acceptable salt thereof in combination with at least one low molecular weight PEO polymer having an approximate molecular weight less than 1,000,000 Da and, optionally, further comprising at least one high molecular weight PEO polymer having an approximate molecular weight of at least 1,000,000 Da; wherein the composition is hot melt extruded or melt granulated, and cured, and wherein the composition exhibits enhanced heat stability while providing resistance to drug segregation between a fines fraction with a particle size of less than about 75 microns and a coarse fraction with a particle size of between about 125 microns and about 250 microns upon grinding; and wherein the resistance to drug segregation results in the percentage of the opioid or pharmaceutically acceptable salt thereof in each of the fines fraction and the coarse fraction being close, characterized by being in the range of about 85% to about 115%, or about 90% to about 110%, or about 95% to about 105%, to that predicted from the composition of the tablet. In some embodiments, the opioid or pharmaceutically acceptable salt thereof is oxycodone hydrochloride.

In some embodiments, the present disclosure includes an extended release oral tablet composition comprising a matrix comprising a therapeutically effective amount of an opioid or a pharmaceutically acceptable salt thereof in combination with at least one low molecular weight PEO polymer having an approximate molecular weight less than 1,000,000 Da and, optionally, further comprising at least one high molecular weight PEO polymer having an approximate molecular weight of at least 1,000,000 Da; wherein the composition is hot melt extruded or melt granulated, and cured, and wherein the composition exhibits enhanced heat stability, and resistance to drug segregation upon grinding measured as a variance of API segregation of less than about 5, or less than about 4.5, or less than about 4, or less than about 3.5, or less than about 3, or less than about 2.5, between a fines fraction with a particle size of less than about 75 microns and a coarse fraction with a particle size of between about 125 microns and about 250 microns. In some embodiments, the opioid or pharmaceutically acceptable salt thereof is oxycodone hydrochloride.

In some embodiments, the present disclosure includes an extended release oral tablet composition comprising a matrix comprising a therapeutically effective amount of an opioid or a pharmaceutically acceptable salt thereof in combination with at least one low molecular weight PEO polymer having an approximate molecular weight less than 1,000,000 Da and, optionally, further comprising at least one high molecular weight PEO polymer having an approximate molecular weight of at least 1,000,000 Da; wherein the composition is hot melt extruded or melt granulated, compressed into a tablet, and the tablet is cured for a period of between about 11 and about 24 hours. In some embodiments, the opioid or pharmaceutically acceptable salt thereof is oxycodone hydrochloride. In some embodiments, the composition maintains an extended release profile of oxycodone, characterized by the percent amount of oxycodone released at 30 minutes, or 60 minutes, or 90 minutes, or 120 minutes, after being subjected to heat pretreatment in an oven at a temperature of about 100° C. for about two hours, with a deviation of no more than about 20%, or about 15%, or about 10%, or about 5%, from an extended release oxycodone oral tablet composition that is not subjected to heat pretreatment.

In some embodiments, the present disclosure includes a method of deterring abuse of an opioid, the method comprising providing an extended release oral tablet composition to a subject in need thereof, wherein the tablet comprises a matrix comprising a therapeutically effective amount of the opioid or pharmaceutically acceptable salt thereof in combination with at least one low molecular weight PEO polymer having an approximate molecular weight less than 1,000,000 Da, a stabilizing agent, and, optionally, further comprising at least one high molecular weight PEO polymer having an approximate molecular weight of at least 1,000,000 Da; wherein the composition is hot melt extruded or melt granulated, compressed into a tablet, and the tablet is cured for a period of between 11 and 24 hours. In some embodiments, the opioid or pharmaceutically acceptable salt thereof is oxycodone hydrochloride.

In some embodiments, the present disclosure includes a method of decreasing abuse potential of an opioid, the method comprising providing an extended release oral tablet composition to a subject in need thereof, wherein the tablet comprises a matrix comprising the opioid or a pharmaceutically acceptable salt thereof in combination with at least one low molecular weight PEO polymer having an approximate molecular weight less than 1,000,000 Da, a stabilizing agent, and, optionally, further comprising at least one high molecular weight PEO polymer having an approximate molecular weight of at least 1,000,000 Da; wherein the composition is hot melt extruded or melt granulated, compressed into a tablet, and the tablet is cured for a period of between 11 and 24 hours. In some embodiments, the opioid or pharmaceutically acceptable salt thereof is oxycodone hydrochloride.

In some embodiments, the present disclosure includes a process for making an extended release, tamper resistant, abuse-deterrent oral tablet composition, comprising: mixing a therapeutically effective amount of an opioid or a pharmaceutically acceptable salt thereof; at least one PEO polymer; optionally, at least one rate limiting polymer; and a stabilizing agent, and hot melt extruding or melt granulating the mixture to form a first component; milling the first component; mixing at least one PEO polymer and a stabilizing agent to form a second component; blending the first component and the second component to form a blended composition; compressing the blended composition to form a tablet; and curing the tablet. In some embodiments, the opioid or pharmaceutically acceptable salt thereof is oxycodone hydrochloride.

In some embodiments, the present disclosure includes an extended release, tamper-resistant, abuse-deterrent oral tablet composition suitable for once or twice daily administration comprising a therapeutically effective amount of an opioid or a pharmaceutically acceptable salt thereof; at least one PEO polymer; a stabilizing agent; and, optionally, at least one rate limiting polymer; wherein an in vitro dissolution profile of the dosage form, characterized by the percent amount of the opioid released at 90 minutes of dissolution, when measured in a USP Apparatus I (basket) at 100 rpm in 900 ml SGF comprising 40% ethanol v/v at 37° C., deviates no more than 20% from corresponding in vitro dissolution measured in a USP Apparatus I (basket) at 100 rpm in 900 ml SGF at 37° C. without ethanol, and wherein the tablet maintains said dissolution profiles after heat pretreatment in an oven at about 100° C. for about two hours or in a microwave at about 1200 W for about 14 minutes. In some embodiments, the opioid or pharmaceutically acceptable salt thereof is oxycodone hydrochloride.

In some embodiments, the present disclosure includes an extended release, tamper-resistant, abuse-deterrent oral tablet composition suitable for once or twice daily administration comprising a therapeutically effective amount of an opioid or a pharmaceutically acceptable salt thereof; at least one PEO polymer; a stabilizing agent; and, optionally, at least one rate limiting polymer; wherein an in vitro dissolution profile of the dosage form, characterized by the percent amount of the opioid released at 90 minutes of dissolution, when measured in a USP Apparatus I (basket) at 100 rpm in 900 ml SGF comprising 40% ethanol v/v at 37° C., deviates no more than 20% from corresponding in vitro dissolution measured in a USP Apparatus I (basket) at 100 rpm in 900 ml SGF at 37° C. without ethanol, and wherein the tablet maintains said dissolution profiles after heat pretreatment in an oven at about 100° C. for about two hours or in a microwave at about 1200 W for about 14 minutes. In some embodiments, the opioid or pharmaceutically acceptable salt thereof is oxycodone hydrochloride.

In some embodiments, the present disclosure includes an oral tablet composition comprising a first component and a second component, wherein the first component comprises an opioid or a pharmaceutically acceptable salt thereof; at least one low molecular weight PEO polymer having a weight average molecular weight of less than 1,000,000 Da; at least one additional nonionic polymer; and a stabilizing agent; and the second component comprises at least one high molecular weight PEO polymer having a weight average molecular weight of at least 1,000,000 Da; and a stabilizing agent, wherein the total combined weight of high and low molecular weight PEO polymers is less than about 65% by weight of the tablet composition, based on the total weight of the composition, wherein the total weight of the high molecular weight PEO polymers is less than about 35% by weight of the tablet composition, based on the total weight of the composition, and wherein the first component is hot melt extruded or melt granulated, milled, and blended with the second component, and the blend is compressed into a tablet, and the tablet is cured. In some embodiments, the opioid or pharmaceutically acceptable salt thereof is oxycodone hydrochloride.

In some embodiments, the present disclosure includes an extended release oral tablet composition comprising a matrix comprising an opioid or a pharmaceutically acceptable salt thereof in combination with at least one low molecular weight PEO polymer having an approximate molecular weight less than 1,000,000 Da, a stabilizing agent, and, optionally, further comprising at least one high molecular weight PEO polymer having an approximate molecular weight of at least 1,000,000 Da; wherein the composition is cured for a period of between 11 and 24 hours, and wherein the composition is formulated to provide resistance to syringeability by limiting the extractability of the opioid or pharmaceutically acceptable salt thereof whereby less than about 20% of the opioid or pharmaceutically acceptable salt thereof is available in syringeable form. In some embodiments, the opioid or pharmaceutically acceptable salt thereof is oxycodone hydrochloride.

### BRIEF DESCRIPTION OF THE DRAWINGS

FIGS. 1a and 1b provide the results of the particle size distributions (PSD) of the ground samples of oxycodone hydrochloride extended release tablets after being subjected to grinding for 5 minutes, comparing tablets according to an embodiment of the present technology (Test) with corresponding tablets of OXYCONTIN® ORT tablets (RLD) (10 mg (FIG. 1a) and 40 mg (FIG. 1b) strengths). The x-axis shows particle sizes in microns.

FIGS. 2a and 2b provide the syringeability results (amount of oxycodone (mg) in syringe after drawing fluid through an 18 gauge (FIG. 2a) or 27 gauge (FIG. 2b) needle into the syringe) from ground oxycodone hydrochloride extended release tablets (40 mg), according to an embodiment of the present technology (Test) compared with OXYCONTIN® ORT tablets (RLD), both without heat pretreatment.

FIG. 3 provides the syringeability results (drawing fluid through a 27 gauge needle into a syringe) from ground oxycodone hydrochloride extended release tablets (10 mg) according to an embodiment of the present technology (Test) compared with OXYCONTIN® ORT tablets (RLD), with and without heat pretreatment (in an oven at 100° C. for two hours).

FIGS. 4a through 4h are directed to the dissolution of oxycodone hydrochloride extended release tablets in SGF.

FIG. 4a demonstrates the dissolution in SGF of oxycodone hydrochloride extended release tablet (40 mg) according to an embodiment of the present technology (Test) compared with OXYCONTIN® ORT tablets (RLD), when not subjected to heat pretreatment.

FIG. 4b demonstrates the dissolution in SGF of oxycodone hydrochloride extended release tablets (40 mg) according to an embodiment of the present technology (Test) compared with OXYCONTIN® ORT tablets (RLD), the dissolution being conducted after heat pretreatment of the tablets in an oven at 100° C. for two hours, or heat pretreatment by microwave irradiation at 1200 W (at 100%) for 11 minutes.

FIGS. 4c and 4d compare dissolution profiles in SGF, with and without heat pretreatment in an oven at 100° C. for two hours, of 10 mg oxycodone hydrochloride extended release tablets according to an embodiment of the present technology (Test) and OXYCONTIN® ORT tablets (RLD), respectively.

FIGS. 4e and 4f compare dissolution profile in SGF, with and without heat pretreatment in an oven at 100° C. for two hours, of 40 mg oxycodone hydrochloride extended release tablets according to an embodiment of the present technology (Test) and OXYCONTIN® ORT tablets (RLD), respectively.

FIGS. 4g and 4h compare dissolution profile in SGF, with and without heat pretreatment in an oven at 100° C. for two hours, of 80 mg oxycodone hydrochloride extended release tablets according to an embodiment of the present technology (Test) and OXYCONTIN® ORT tablets (RLD), respectively.

FIGS. 5a through 5g are directed to the dissolution of oxycodone hydrochloride extended release tablets in SGF containing 40% v/v of ethanol.

FIG. 5a demonstrates the dissolution properties of oxycodone hydrochloride extended release tablets (40 mg) according to an embodiment of the present technology (Test) compared with OXYCONTIN® ORT tablets (RLD), using 40% v/v of ethanol-induced dose dumping dissolution method, before and after heat pretreatment in an oven at 100° C. for two hours, or heat pretreatment by microwave irradiation at 1200 W for 14 minutes.

FIGS. 5b and 5c compare dissolution profiles in SGF containing 40% v/v of ethanol, with and without heat pretreatment in an oven at 100° C. for two hours, of 10 mg oxycodone hydrochloride extended release tablets according to an embodiment of the present technology (Test) and OXYCONTIN® ORT tablets (RLD), respectively.

FIGS. 5d and 5e compare dissolution profile in SGF containing 40% v/v of ethanol, with and without heat pretreatment in an oven at 100° C. for two hours, of 40 mg oxycodone hydrochloride extended release tablets according to an embodiment of the present technology (Test) and OXYCONTIN® ORT tablets (RLD), respectively.

FIGS. 5f and 5g compare dissolution profile in SGF containing 40% v/v of ethanol, with and without heat pretreatment in an oven at 100° C. for two hours, of 80 mg oxycodone hydrochloride extended release tablets according to an embodiment of the present technology (Test) and OXYCONTIN® ORT tablets (RLD), respectively.

FIGS. 6a through 6e are directed to extraction of oxycodone hydrochloride extended release tablets.

FIG. 6a demonstrates results with cut tablets (four pieces) from extraction studies of oxycodone hydrochloride extended release tablets (40 mg) according to an embodiment of the present technology (Test) compared with OXYCONTIN® ORT tablets (RLD), after extraction with different solvents for five hours.

FIGS. 6b and 6c demonstrate the results with cut tablets (four pieces) from extraction studies (USP Apparatus II (Paddle); 500 ml volume; 150 rpm) of oxycodone hydrochloride extended release tablets (40 mg) according to an embodiment of the present technology (Test) compared with OXYCONTIN® ORT tablets (RLD), in water (FIG. 6b) and in 50% v/v water:ethanol (FIG. 6c), with and without heat pretreatment (heat pretreatment preceding tablet cutting; either heat pretreatment in an oven at 100° C. for two hours, or heat pretreatment by microwave irradiation at 1200 W for 14 minutes).

FIGS. 6d and 6e show the amount of oxycodone hydrochloride withdrawn at 10 minutes from an aqueous solution (10 ml) made at room temperature (FIG. 6d) and at 90° C. (FIG. 6e), of Test Product and OXYCONTIN® ORT tablets (RLD), with and without heat pretreatment, into a syringe with 27 gauge needle.

FIGS. 7a through 7c demonstrate the molecular weight distributions of PEO polymers present in oxycodone hydrochloride extended release tablets (40 mg) according to an embodiment of the present technology, with and without heat pretreatment (in an oven at 100° C. for two hours) (FIG. 7a), with and without heat pretreatment (microwave irradiation at 1200 W for 14 minutes) (FIG. 7b); and PEO polymers present in OXYCONTIN® (RLD) with and without heat pretreatment (microwave irradiation at 1200 W for 14 minutes) (FIG. 7c); all compared with PEO standards (POLYOX™)

FIG. 8 compares human plasma concentration profiles of oxycodone hydrochloride ER tablets of the present technology (Test) with OXYCONTIN® ORT tablets (RLD) (40 mg) under fed conditions.

FIG. 9 compares human plasma concentration profiles of oxycodone hydrochloride ER tablets of the present technology (Test) with OXYCONTIN® ORT tablets (RLD) (40 mg) under fasting conditions.

### DEFINITIONS

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “pharmaceutically acceptable” means a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the FDA.

As used herein, the terms “active agent,” “active ingredient,” “pharmaceutical agent,” and “drug” refer to any material that is intended to produce a therapeutic, prophylactic, or other intended effect. These terms with respect to specific agents include all pharmaceutically active agents, all pharmaceutically acceptable salts thereof, and all complexes, stereoisomers, crystalline forms, cocrystals, ether, esters, prodrugs, hydrates and solvates thereof, and mixtures thereof, which produce the intended effect.

As used herein, the phrase “therapeutically effective amount” means that amount that provides the specific pharmacological response for which the agent is administered to a subject in need of such treatment, for whatever reason. It is emphasized that a therapeutically effective amount will not always be effective in treating the target conditions/diseases, even though such amount is deemed to be a therapeutically effective amount by those of skill in the art. For illustration only, exemplary doses and therapeutically effective amounts are provided below with reference to adult human subjects. Those skilled in the art can adjust such amounts in accordance with standard practices as needed to treat a specific subject and/or condition/disease.

As used herein, the term “pharmaceutically acceptable salts” should be ascribed its customary meaning, and includes, but is not limited to, inorganic acid salts such as hydrochloride, hydrobromide, sulfate, phosphate, and the like; organic acid salts such as formate, acetate, trifluoroacetate, maleate, tartrate, and the like; sulfonates such as methanesulfonate, benzenesulfonate, p-toluenesulfonate, and the like; amino acid salts such as arginate, asparaginate, glutamate, and the like; metal salts such as sodium salt, potassium salt, cesium salt, and the like; alkaline earth metals such as calcium salt, magnesium salt, and the like; and organic amine salts such as triethylamine salt, pyridine salt, picoline salt, ethanolamine salt, triethanolamine salt, discyclohexylamine salt, N,N′-dibenzylethylenediamine salt, and the like.

The term “patient” means a subject who has presented a clinical manifestation of a particular symptom or symptoms suggesting the need for treatment, who is treated preventatively or prophylactically for a condition, or who has been diagnosed with a condition to be treated.

The term “subject” is inclusive of the definition of the term “patient.” As used herein “a subject in need” of treatment by the methods described herein includes any subject suffering from or at risk of developing pain as described herein. The subject can be any mammal, including humans, horses, cats, and dogs. In particular embodiments, the subject is a human.

As used herein, the term “extended release” denotes a pharmaceutical composition (e.g., a pharmaceutical composition formulation) that provides release of an active drug substance from the composition for an extended period of time, i.e., for a longer period of time than that seen with an immediate release formulation. In certain circumstances, the term “extended release” is used to designate a release at a desired rate during a predetermined release period. Additional or alternative terms, such as, for example, “modified,” “delayed,” “sustained,” or “prolonged” release may be used, in certain embodiments, as synonyms to the term “extended release.”

The term “immediate release,” as used herein, denotes a pharmaceutical composition that releases the active drug substance (not less than 80% release) within 5-60 minutes, e.g., 15 minutes, 20 minutes, 30 minutes, etc., when subjected to dissolution testing according to USP 37, NF 32, in commonly used USP Apparatus I or II.

The term “stabilizing agent” means a compound or composition that serves to minimize or reduce deterioration of one or more properties of a pharmaceutical composition of the present embodiments, especially where the one or more properties may serve to create or enhance the abuse-deterrent properties of the pharmaceutical composition.

The term “oxidative stabilizing agent” means a stabilizing agent that serves to minimize or reduce the oxidative degradation and loss of viscosity that would otherwise occur when a heat-labile gelling agent, such as PEO, is subjected to heat. The oxidative stabilizing agent may be heat-resistant, meaning it does not decompose under hot melt extruding, melt granulation, and/or curing conditions, and/or other heat-related conditions as described, e.g., in the present embodiments. The oxidative stabilizing agent may suppress oxidative degradation of oxidative degradable matrix materials such as PEO polymer and oxidation sensitive drugs in pharmaceutical dosage forms.

The term “curing” refers to subjecting a pharmaceutical composition to an elevated temperature, e.g., at least as high as the melting temperature of at least one of the PEO polymers contained in the pharmaceutical composition, for a specified period of time.

The terms “tampering” and “tamper” refer to manipulation by mechanical, thermal, and/or chemical means to obtain a solution or particulate material of a drug. Examples of tampering include, but are not limited to, crushing, grinding, grating, cutting, and other mechanical methods of particle-size reduction. Also included are crisping, heating, irradiating, and other methods of deteriorating a functional characteristic associated with one or more components within a pharmaceutical composition. As used herein, the term “crisping” means heating of the dosage form (e.g., microwave irradiation, oven, hot plate, lighter, candle) to degrade the abuse-deterrent properties of the dosage form.

The term “heat pretreatment” refers to subjecting a pharmaceutical composition to heating conditions, e.g., heating in an oven or in a microwave, before manipulation by mechanical and/or chemical means. The term “heat pretreatment” does not include curing.

The term “heat stability” refers to the stability of a pharmaceutical composition, as measured by resistance to a heat pretreatment-induced increase in, for example, syringeability, extractability, dissolution, and/or alcohol-dose dumping, upon mechanical and/or chemical manipulation, after subjecting the composition to heat pretreatment. Mechanical and/or chemical manipulations can include, but are not limited to, crushing, grinding, grating, cutting, milling, and/or alcohol-dose dumping. The term “enhanced heat stability” is a comparative term referring to a superior form of the heat stability of a pharmaceutical formulation, as defined above. Without being bound by a theory, it is believed such superior heat stability is the result of several factors, including, but not limited to, the stabilization of PEO polymers in the presence of a suitable antioxidant that can withstand elevated temperatures. For example, such antioxidant provides enhanced heat stability to the PEO polymer during the hot melt extrusion or melt granulation process, and/or during the curing process, and/or the antioxidant prevents oxidative degradation of oxycodone, and/or the antioxidant prevents auto-oxidation of the PEO polymer.

The term “resistance to drug segregation” refers to the property of a pharmaceutical composition resulting in decreased or negligible drug segregation. For example, a pharmaceutical composition exhibits resistance to drug segregation when the percentage of drug content in the fines fraction (and/or in the coarse fraction) of a ground tablet is close to that predicted from the composition of the tablet itself. For example, the percentage of drug content in the fines (or coarse) fraction in a pharmaceutical composition exhibiting a resistance to drug segregation can be in the range of about 80% to about 130% to that predicted from the composition of the tablet (e.g., about 100%). More specifically, the percentage of drug content in a fines (or coarse) fraction can be in the range of about 80% to about 130%, about 85% to about 125%, about 85% to about 120%, about 85% to about 115%, about 90% to about 110%, about 95% to about 105%, to that predicted from the composition of the tablet.

The term “abuse-deterrent” refers to oral dosage forms that reduce the potential for improper administration of the drugs contained therein, but that deliver a therapeutically effective dose when administered as directed. Improper administration includes tampering with the dosage form and/or administering the drug by any route other than instructed. For example, and without limitation, improper administration includes snorting, administration after heat treatment (e.g., heat pretreatment), oral administration after crushing, or parenteral administration after extraction with a solvent such as water, ethanol, isopropanol, acetone, acetic acid, vinegar, carbonated beverages, and the like, and combinations thereof.

The term “melting temperature” refers to a temperature which is at least as high as the melting point (e.g., 65-70° C.) of the lower molecular weight POLYOX® in the drug formulation.

The term “grinding” refers to a process of reducing one or more tablets into small fragments, e.g., in the form of powder, following a specific grinding pattern, e.g., two min grinding/one min rest/two min grinding, using electrical (e.g., coffee grinder or IKA grinder) grinding means.

The term “coarse fraction,” as used herein, refers to a fraction of particulates with particle sizes of at least about 125 microns, for example, between about 125 microns and about 250 microns.

The term “fines fraction” refers to a fraction of particulates with particle sizes of less than about 125 microns, for example, less than about 75 microns.

The term “resistant to alcohol extraction” is used to refer to dosage forms that at least fulfill the condition that in vitro dissolution, when measured in a USP Apparatus I (basket) at 100 rpm in 900 ml simulated gastric fluid comprising 40% ethanol (v/v) at 37° C., is provided that is characterized by a percent amount of active released at 30 minutes of dissolution that deviates no more than 20% from the corresponding in vitro dissolution measured in a USP Apparatus I (basket) at 100 rpm in 900 ml simulated gastric fluid at 37° C. without ethanol.

The term “rate limiting polymer” refers to a polymer that can modify the release rate of the drug contained therein, e.g., in a pharmaceutical formulation containing the polymer.

### DETAILED DESCRIPTION OF THE INVENTION

### 1. Abuse-Deterrent Formulations

The present technology generally provides abuse-deterrent oral solid pharmaceutical formulations providing an extended release of an active agent, such as an opioid.

In some embodiments, the present technology is a solid pharmaceutical composition, comprising a cured blend of a melt-extruded/melt granulated first component and a second component, wherein the melt-extruded/melt granulated first component comprises an opioid or a pharmaceutically acceptable salt thereof, at least one PEO polymer, and an oxidative stabilizing agent; and the second component comprises at least one PEO polymer and an oxidative stabilizing agent.

In some embodiments, the first component further comprises an additional nonionic pH-independent polymer. In some embodiments, the first component further comprises a plasticizer.

In some embodiments, the first component further comprises a complexing agent.

In some embodiments, the first component further comprises a lubricant. In some additional or alternative embodiments, the blend of the first and second components further comprises a lubricant or glidant, inorganic or organic fillers, surfactants, disintegrants, or combinations thereof.

In some embodiments, the solid pharmaceutical formulation is a compressed tablet. In specific embodiments, the tablet is coated with a coating. In some embodiments, the coating is a cosmetic coating, a coating that modifies the release of the opioid, including but not limited to a coating that dissolves under acidic pH, enteric coating, or combinations thereof.

In some embodiments, the solid pharmaceutical formulation further includes additional abuse-deterrent agents, including bittering agents, irritants, or dyes.

### Drugs of Abuse

The pharmaceutical formulations of the present disclosure generally comprise at least one drug susceptible to abuse, e.g., an opioid. In some embodiments, the opioid is selected from the group comprising, but not limited to, morphine, oxycodone, hydrocodone, hydromorphone, norhydrocodone, oxymorphone, noroxycodone, and morphine-6-glucuronide, along with hydrates, solvates, cocrystals, prodrugs, and pharmaceutically acceptable salts thereof.

In some embodiments, the drugs susceptible to abuse include, but are not limited to, opioid analgesics, benzodiazepines, tranquilizers, stimulants, psychoactive drugs, and other drugs that may cause euphoria and/or psychological and/or physical dependence. In some embodiments, the drugs susceptible to abuse include, but are not limited to, methylphenidate, fentanyl, carfentanil, remifentanil, sufentanil, etorphine, ohmefentanyl, amphetamines, methamphetamine, ephedrine, pseudoephedrine, pseudoephedrine HCl, pseudoephedrine sulfate, alfentanil, alphaprodine, buprenorphine, butorphanol, clonitazene, dextromoramide, dextropropoxyphene, dezocine, ethylmorphine, dihydrocodeine, dihydromorphine, codeine, benzylmorphine, desomorphine, diphenoxylate, diprenorphine, levomethadryl, levomethadone, levorphanol, lofentanil, meperidine, methadone, methylmorphine, nalbuphine, nicomorphine, nicomorphine, normethadone, norpipanone, pentazocine, pethidine, propoxyphene, tapentadol, tilidine, tramadol, norpseudoephedrine, and phenylpropanolamine, including salts, derivatives, analogs, homologues, polymorphs thereof, and mixtures of any of the foregoing.

In certain embodiments, the opioid is oxycodone, hydromorphone, oxymorphone, and hydrocodone, or a pharmaceutically acceptable salt thereof such as, e.g., the hydrochloride. In some embodiments, the tablet comprises from about 5 mg to about 500 mg oxycodone, from about 1 mg to about 100 mg hydromorphone or from about 5 mg to about 500 mg oxymorphone, each of which may be in the form of a free base; or as a pharmaceutically acceptable salt, hydrate, solvate, or cocrystal; or in a derivative form including, but not limited to, prodrugs. In some embodiments, the tablet comprises, e.g. about 5 mg, about 7.5 mg, about 10 mg, about 15 mg, about 20 mg, about 30 mg, about 40 mg, about 45 mg, about 60 mg, or about 80 mg, about 90 mg, about 120 mg, or about 160 mg, and ranges between any two of these values (including endpoints) of oxycodone in the form of a free base; or as a pharmaceutically acceptable salt, hydrate, solvate, or cocrystal; or in a derivative form including, but not limited to, prodrugs. In other embodiments, the tablet comprises, e.g., about 5 mg, about 7.5 mg, about 10 mg, about 15 mg, about 20 mg, about 30, mg, about 40 mg, about 45 mg, about 60 mg, about 80 mg, about 90 mg, about 120 mg, or about 160 mg, and ranges between any two of these values (including endpoints) of oxymorphone in the form of a free base; or as a pharmaceutically acceptable salt, hydrate, or solvate; or in a derivative form including, but not limited to, prodrugs. In other embodiments, the tablet comprises, e.g., about 2 mg, about 4 mg, about 8 mg, about 12 mg, about 16 mg, about 24 mg, about 32 mg, about 48 mg, or about 64 mg, and ranges between any two of these values (including endpoints) of hydromorphone in the form of a free base; or as a pharmaceutically acceptable salt, hydrate, or solvate; or in a derivative form including, but not limited to, prodrugs. The above weights are based on the weight of the active compound in free base form.

In some embodiments, the tablet includes about 1 mg to about 200 mg oxycodone hydrochloride. In some embodiments, the tablet includes about 10 mg, about 15 mg, about 20 mg, about 30 mg, about 40 mg, about 60 mg, or about 80 mg, and ranges between any two of these values (including endpoints) of oxycodone hydrochloride.

### Polyethylene Oxide

The solid pharmaceutical formulations of the present disclosure comprise a PEO polymer. PEO polymers (PEOs) are linear polydisperse nonionic polymers composed of repeating units of ethylene oxide. Their chemical formula is HO[CH₂CH₂O]ₙH, where n represents the average number of oxyethylene groups. Depending on preparation method, high molecular weight PEO may have one terminal methyl group.

PEOs that are suitable for use in the tablets of the present technology are those having a weight average molecular weight in the range of about 200,000 to about 10,000,000 Da, for example in the range of about 250,000 to about 10,000,000 Da, in the range of about 300,000 to about 9,000,000 Da, in the range of about 350,000 to about 9,000,000 Da, or in the range of about 900,000 to about 7,000,000 Da, based on rheological measurements. In certain embodiments, the PEOs that are suitable for use in the compositions disclosed herein are those having a weight average molecular weight of about 900,000 Da, about 1,000,000 Da, about 2,000,000 Da, about 3,000,000 Da, about 4,000,000 Da, about 5,000,000 Da, about 6,000,000 Da, about 7,000,000 Da, about 8,000,000 Da, or about 9,000,000 Da, and ranges between any two of these values (including endpoints).

In some embodiments, the oral tablets comprise at least two different PEO polymers having different weight average molecular weights. In some embodiments, the first component and the second component comprise PEO polymers having different weight average molecular weights. In some embodiments, the first component comprises at least one low molecular weight polyethylene oxide (PEO) polymer having a weight average molecular weight of less than 1,000,000 Da, and the second component comprises at least one high molecular weight PEO polymer having a weight average molecular weight of at least 1,000,000 Da. In some embodiments, the tablet comprises a dosage form having less than about 80% by weight, less than about 75% by weight, less than about 70% by weight, less than about 69% by weight, less than about 68% by weight, less than about 67% by weight, less than about 66% by weight, less than about 65% by weight, less than about 64% by weight, less than about 63% by weight, less than about 62% by weight, less than about 61% by weight, less than about 60% by weight, less than about 55% by weight, or less than about 50% by weight, based on the total weight of the composition of the total combined weight of the high and low molecular weight polyethylene oxides. In some embodiments, the tablet comprises a dosage form having less than about 50% by weight, less than about 45% by weight, less than about 40% by weight, less than about 35% by weight, less than about 34% by weight, less than about 33% by weight, less than about 32% by weight, less than about 31% by weight, or less than about 30% by weight, based on the total weight of the composition of the total weight of the high molecular weight polyethylene oxides. In some embodiments, the first component and the second component comprise PEO polymers having the same average molecular weight.

Exemplary polyethylene oxide polymers include POLYOX™ WSR N-80, POLYOX™ WSR N-750, POLYOX™ WSR N-3000, POLYOX™ WSR-205, POLYOX™ WSR N-1105, POLYOX™ WSR N-12K, POLYOX™ WSR N-60K, POLYOX™ WSR N-301, POLYOX™ WSR Coagulant, POLYOX™ WSR N-303. The exemplary polyethylene oxide polymers provide different viscosities in an aqueous solution.

A person of skill in the art will appreciate that the appropriate PEO polymer can be chosen depending on the desired viscosity and hardness of the oral tablet.

In some embodiments, the concentration of PEO in the solid pharmaceutical composition is in the range of about 20 to about 90% w/w. In some embodiments, the concentration of PEO in the first component is in the range of about 5 to about 99.9% w/w, with respect to the first component, such as from about 10 to about 99.9% w/w, such as from about 10 to about 98% w/w, such as from about 20 to about 98% w/w, such as from about 30 to about 98% w/w, such as from about 40 to about 98% w/w, such as from about 50 to about 98% w/w, such as from about 60 to about 98% w/w, such as from about 70 to about 98% w/w. In particular embodiments, the concentration of PEO in the first component is in the range of about 10 to about 60% calculated as w/w % of the first component, such as about 10%, about 15%, about 20%, about 25%, about 30%, about 35%, about 40%, about 45%, about 50%, about 55%, or about 60% w/w, and ranges between any two of these values (including endpoints).

In some embodiments, the concentration of PEO in the second component is in the range of about 5 to about 99.9% w/w, with respect to the second component, such as from about 10 to about 99.9%, from about 10 to about 98%, from about 20 to about 98%, from about 30 to about 98%, from about 40 to about 98%, from about 50 to about 98%, from about 60 to about 98%, or from about 70 to about 98% w/w. In particular embodiments, the concentration of PEO in the second component is in the range of about 10 to about 60% calculated as w/w % of the second component, such as about 10%, about 15%, about 20%, about 25%, about 30%, about 35%, about 40%, about 45%, about 50%, about 55%, or about 60% w/w, and ranges between any two of these values (including endpoints).

### Rate Limiting Polymer

In some embodiments, the dosage form includes a polymer that can modify the release rate of the drug contained therein. Examples of polymers that can be utilized to modify the release rate of the drug include pharmaceutically acceptable cellulosic polymers, including but not limited to cellulose esters, cellulose diesters, cellulose triesters, cellulose ethers, cellulose ester-ethers, cellulose acylates, cellulose diacylates, cellulose triacylates, cellulose acetates, cellulose diacetates, cellulose triacetates, cellulose acetate propionates, cellulose acetate butyrates, and mixtures thereof. In some embodiments, the cellulosic polymer is an alkyl cellulosic polymer such as hydroxypropyl methylcellulose (HPMC), hydroxypropyl cellulose (HPC), or ethylcellulose. In some embodiments, the rate limiting polymers include polyvinyl pyrrolidone (PVP); polyvinyl acetate (PVA); a polymer that is a mixture of PVA, PVP, sodium lauryl sulfate, and silica; and mixtures thereof.

Other rate limiting polymers include pharmaceutically acceptable acrylic polymers selected without limitation from acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid) (anhydride), methyl methacrylate, polymethacrylate, poly(methyl methacrylate), poly(methyl methacrylate) copolymer, polyacrylamide, aminoalkyl methacrylate copolymer, poly(methacrylic acid anhydride), glycidyl methacrylate copolymers, and mixtures of any of the foregoing. Preferably, the acrylic polymer is a neutral acrylic polymer (e.g., Eudragit NE 30 D®, Eudragit NE 40 D® or Eudragit NM 30 D®).

### Stabilizing Agent

The oral tablets of the present disclosure comprise a stabilizing agent. In some embodiments, the stabilizing agent is an oxidative stabilizing agent, for example, an antioxidant suitable for use in a pharmaceutical composition. In some embodiments, the stabilizing agent is not subject to degradation under one or more of the hot melt extrusion, melt granulation, and curing conditions of the particular embodiments herein. In some embodiments, the stabilizing agent is selected from the group consisting of d-alpha-tocopherol, polyethylene glycol 1000 succinate, ascorbic acid, dl-alpha-tocopherol, dl-alpha-tocopherol acetate, alpha-tocopherol, vitamin E, sodium citrate, citric acid, tocopherols, butylated hydroxy toluene (BHT), and butylated hydroxyanisole (BHA). In particular embodiments, the stabilizing agent is selected from the group consisting of d-alpha-tocopherol, polyethylene glycol 1000 succinate, ascorbic acid, dl-alpha-tocopherol, dl-alpha-tocopherol acetate, alpha-tocopherol, vitamin E, sodium citrate, and citric acid. In particular embodiments, the stabilizing agent is dl-alpha-tocopherol.

In some embodiments, the stabilizing agent is present in the first component at a concentration of about 0.01% w/w to about 3.0% w/w, with respect to the first component. In particular embodiments, the stabilizing agent is present in the first component at a concentration of about 0.02% w/w, about 0.04% w/w, about 0.06% w/w, about 0.08% w/w, about 0.1% w/w, about 0.2% w/w, about 0.4% w/w, about 0.6% w/w, about 0.8% w/w, about 1% w/w, about 1.5% w/w, about 2% w/w, about 2.5% w/w, or about 3% w/w, and ranges between any two of these values (including endpoints).

In some embodiments, the stabilizing agent is present in the second component, and in an above described amount. In other additional or alternative embodiments, the stabilizing agent is present in the blend of the first and the second components, and in an above described amount.

### Polymeric Excipients

In some embodiments, the oral tablet of the present technology includes a nonionic pH-independent polymer in addition to PEO. In some embodiments, the nonionic pH-independent polymer is a cellulosic polymer such as ethylcellulose, hydroxypropyl methylcellulose (HPMC), hydroxypropylcellulose, hydroxymethylcellulose, hydroxyethylcellulose, cellulose acetate, or mixtures thereof. In some embodiments, the nonionic pH-independent polymer is a polyvinylpyrrolidone (PVP), a PVP-based polymer, a polyvinylacetate (PVA), a PVA-based polymer, or a blend, such those marketed under the trade name KOLLIDON® (e.g., KOLLIDON® SR). In particular embodiments, the nonionic pH-independent polymer is HPMC. In some embodiments, the nonionic pH-independent polymer is present in the first component at a concentration of about 1.5% w/w to about 20% w/w, with respect to the first component. In particular embodiments, the nonionic pH independent polymer is present at a concentration of about 2% w/w, about 4% w/w, about 6% w/w, about 8% w/w, about 10% w/w, about 12% w/w, about 14% w/w, about 16% w/w, about 18% w/w, or about 20% w/w, and ranges between any two of these values (including endpoints), with respect to the first component.

### Complexing Agent

In some embodiments, the oral tablet of the present technology includes a complexing agent. Without being bound by theory, it is hypothesized that the complexing agent forms a complex with the drug by trapping the drug in its pores and prevents release upon tampering by an abuser. In some embodiments, the complexing agent is included in the first component. In other additional or alternative embodiments, the complexing agent is included in the second component or in the blend of the first and second components. In particular embodiments, the complexing agent is colloidal silica or silicon dioxide. In some embodiments, the complexing agent is colloidal silica or silicon dioxide having a particle size of less than 20 microns.

In some embodiments, the complexing agent is present in the first component at a concentration of about 0.2% w/w to about 20% w/w, with respect to the first component. In particular embodiments, the complexing agent is present at a concentration of about 0.2% w/w, about 1% w/w, about 2% w/w, about 4% w/w, about 6% w/w, about 8% w/w, about 10% w/w, about 12% w/w, about 14% w/w, about 16% w/w, about 18% w/w, or about 20% w/w, and ranges between any two of these values (including endpoints). In other additional or alternative embodiments, the complexing agent is included in the second component or in the blend of the first and second components, and in an above described amount.

### Plasticizer

In some embodiments, the oral tablet of the present technology includes a plasticizer. Without being bound by theory, it is hypothesized that the plasticizer confers greater crush resistance to the tablet. Without being bound by theory, it is also hypothesized that the plasticizer can deter abuse because it can act as a tissue irritant if administered with an opioid. In some embodiments, the plasticizer can be included in the first component, or in the second component, or in the blend of the first component and the second component. Examples of plasticizers include, but are not limited to, dibutyl sebacate, glycerol, diethylene glycol monoethyl ether, diethyl phthalate, glycol triacetate, polyoxyethylene sorbitan monolaurate, polyethylene glycol, propylene glycol, glycerol, triacetin, tributyl citrate, and triethyl citrate and mixtures thereof. In a particular embodiment, the plasticizer is triethyl citrate.

In some embodiments, the plasticizer is present in the composition at a concentration of about 0.5% w/w to about 15% w/w, with respect to the oral tablet. In particular embodiments, the plasticizer is present at a concentration of about 0.5% w/w, about 1% w/w, about 2% w/w, about 4% w/w, about 6% w/w, about 8% w/w, about 10% w/w, about 12% w/w, or about 15% w/w, and ranges between any two of these values (including endpoints).

### Other Excipients

In some embodiments, the oral tablet of the present technology can include other excipients that improve functionality and processability of dosage forms. For example, the oral tablet can include excipients such as lubricants and glidants, inorganic or organic fillers, surfactants, etc. One or more extragranular materials, such as lubricants or glidants, can be used to keep the multiparticulates from sticking together. Examples of suitable materials for this purpose include, but are not limited to, magnesium stearate, zinc stearate, colloidal silicone dioxide, talc, starch, calcium stearate, hydrogenated vegetable oils, stearic acid, sodium stearyl fumarate, sodium benzoate, sodium acetate, leucine, sodium oleate, sodium lauryl sulfate, magnesium lauryl sulfate and polyethylene glycol.

Disintegrants can be added to pharmaceutical formulations in order to facilitate “breakup” or disintegration after administration. Materials used for this purpose include starches, clays, celluloses, aligns, gums, and cross-linked polymers.

One or more surfactants may also be added to the final dosage form to modulate the release of drug from the solid pharmaceutical formulation. Examples include, but are not limited to, lecithin, sodium dodecyl sulfate, poloxamer, polyethoxylated castor oil (e.g., KOLLIPHOR EL® (CREMOPHOR EL®)), polysorbates, and polyoxyglycerides.

In some embodiments a sustained release matrix can also be prepared by a melt granulation technique involving a normally solid hydrophobic binding material, e.g., wax, and incorporating a powdered drug therein.

Hydrophobic binder material is selected from natural and synthetic waxes, fatty acids, fatty alcohols, and mixtures of the same. Examples include, bees wax, carnauba wax, castor wax, glycowax, stearic acid, and stearyl alcohol. In certain embodiments, a combination of two or more hydrophobic binder materials are included in the matrix formulations.

In addition to the additives above, coloring and flavoring agents may also be incorporated into the solid pharmaceutical formulation.

In some embodiments, the solid pharmaceutical formulation further includes additional abuse-deterrent agents, including bittering agents, irritants, or dyes. The bittering agent includes any pharmaceutically acceptable bitter substance that creates a bitter taste or side effect when administered nasally (snorted), orally, buccally, or sublingually. Such agents include, but are not limited to, sucrose octaacetate, denatonium saccharide, denatonium benzoate, caffeine, quinine (or a quinine salt such as quinine sulfate), bitter orange peel oil, and other botanical extract ingredients, such as pepper extract (Cubeb), capsicum, and the like.

An indicator dye useful in the present technology includes any dye that is pharmaceutically acceptable and that is capable of providing an intense, bright color on the nose, mouth and hands after a pharmaceutical formulation containing the dye is crushed or dissolved. The bright color also can have a psychologically deterrent effect on intravenous abusers. Such dyes include, but are not limited to allura red, amaranth, brilliant blue, canthaxanthin, carmine, carmoisine, carotene, curcumin, erythrosine, green S, indigo carmine, iron oxide black, iron oxide red, iron oxide yellow, patent blue, phloxine O, ponceau 4R, quinoline yellow, riboflavin, sunset yellow, tartrazine, titanium dioxide, vegetable carbon black, and other natural colors such as annatto, beet, black carrot, black currant, caramel, carmine, carmine lake, chlorophyll, cochineal, elderberry, grapeskin/grape juice, malt, paprika, red cabbage, turmeric, and anthocyanins.

In yet other embodiments, the solid pharmaceutical formulation can include an irritant, such as, capsaicin, a capsaicin analog with similar type properties as capsaicin, and the like. Some capsaicin analogues or derivatives include for example and without limitation, resiniferatoxin, tinyatoxin, heptanoylisobutylamide, heptanoyl guaiacylamide, other isobutylamides or guaiacylamides, dihydrocapsaicin, homovanillyl octylester, nonanoyl vanillylainide, or other compounds of the class known as vanilloids.

### Opioid Antagonist

In some embodiments, the oral tablet of the present technology can include an opioid antagonist. Examples of opioid antagonists include naltrexone, 6-beta-naltrexol, nalbuphine, nalmefene, naloxone, cyclazocine, levallorphan, cyclorphan, and oxilorphan. Without being bound by theory, it is believed that upon tampering by an abuser, the opioid antagonist will be extracted along with opioid and prevent the isolation of opioid as a single entity to be abused.

### Coatings

Individual tablets can also include a film coating, e.g., to enhance cosmetic appearance and/or to reduce tackiness. Examples of materials to be utilized as a film coat include hydroxypropyl methylcellulose, polyvinyl alcohol, lactose, or a mixture thereof. The film coat can be an outer coating, an outer coating along with a release-modifying coating, or an intermediate layer between a substrate and a release modifying coating.

In other embodiments, the coating can include a cationic polymer, which inhibits drug release when the dosage form is chewed, but dissolves at gastric pH. Examples of a cationic polymer include, but are not limited to, Eudragit E PO.

### 2. Methods of Making

In some embodiments, the present technology includes a process of making an oral tablet comprising:

mixing an opioid or a pharmaceutically acceptable salt thereof, at least one PEO polymer, at least one additional nonionic pH-independent polymer (optional), and a stabilizing agent;

melt extruding the mixture to form a first component;

milling the melt-extruded first component;

mixing at least one PEO polymer and a stabilizing agent to form a second component;

blending the ground first component and the second component with a lubricant to form a blended composition;

compressing the blended composition to form a tablet; and

curing the tablet.

In some embodiments, the present technology includes a process of making an oral tablet comprising:

optionally mixing the opioid or a pharmaceutically acceptable salt thereof with a complexing agent by shearing and optional granulation;

preparation of the first component by blending an opioid or a pharmaceutically acceptable salt thereof or an opioid-complexing agent complex with at least one PEO polymer; mixing a stabilizing agent with an optional solvent such as ethanol and adding the stabilizing agent to the opioid-PEO polymer blend while mixing. Optionally, additional excipients, such as a nonionic polymer, ion-exchange resin, plasticizer, disintegrants, or lubricants, or combinations thereof, are included into the first component blend;

hot melt extrusion of first component, followed by cooling (e.g., with liquid nitrogen, dry ice, or any other cooling mechanism known to one in the art) and preparation (e.g., milling) of first component granulate;

preparation of the second component by blending at least one PEO polymer;

mixing a stabilizing agent with an optional solvent such as ethanol and adding the stabilizing agent to the PEO polymer blend while mixing;

the first component and the second component are blended with each other and optionally additional excipients, such as plasticizers, disintegrants, or lubricants, or combinations thereof;

compression of the final blended composition into tablets;

curing of the tablets at a sufficient temperature to fuse the first and second components into a cured form;

optionally coating the tablets to obtain an extended release oral dosage form.

A sustained release matrix can also be prepared by, for example, a melt granulation technique. Generally, melt granulation technique involves melting a normally solid hydrophobic binding material, e.g., wax, and incorporating a powdered drug therein. To obtain a sustained release dosage form, it may be necessary to incorporate a hydrophobic sustained release material, e.g., ethyl cellulose or a water insoluble acrylic polymer, into the molten wax hydrophobic binder material. Examples of sustained release formulations prepared via melt granulation techniques are found in U.S. Pat. No. 4,861,598, which is incorporated by reference in its entirety.

### Hot Melt Extrusion

At least a portion of the pharmaceutical compositions embodied herein can be processed through a hot melt extrusion step, or at least subjected to elevated temperature and pressure. Hot melt extrusion is well known in the art, and the specific embodiments contained herein are not meant to be limiting. The skilled artisan would recognize that the hot melt extrusion parameters may be varied depending on, e.g., convenience, scale, etc.

Typical hot melt extrusion systems suitable for use in the present technology include a suitable extruder drive motor having variable speed and constant torque control, start-stop controls, and ammeter. In addition, the system will include a temperature control console that includes temperature sensors, cooling means, and temperature indicators throughout the length of the extruder. In addition, the system will include an extruder, such as twin-screw extruder, which consists of two counter-rotating intermeshing screws enclosed within a cylinder or barrel having an aperture or die at the exit thereof. The feed materials for the first component enter through a feed hopper and are moved through the barrel by the screws and forced through the die into strands, which are thereafter conveyed, such as by a continuous movable belt, to allow for cooling and being directed to a pelletizer or other suitable device to render the extruded ropes into the multiparticulate system. The pelletizer can consist of rollers, fixed knife, rotating cutter, and the like. Suitable instruments and systems are available from distributors such as Leistritz Advanced Technologies Corp. (Allendale, N.J.); C. W. Brabender Instruments, Inc. (South Hackensack, N.J.). Other suitable apparatuses will be apparent to those of ordinary skill in the art.

A further aspect of the invention is related to the preparation of the hot melt extruded first component as set forth above in a manner that controls the amount of air included in the extruded product. Without being bound by theory, it is believed that by controlling the amount of air included in the extrudate, the release rate of the therapeutically active agent from the oral tablet can be altered significantly. In certain embodiments, it has been surprisingly found that the pH dependency of the extruded product can be altered as well.

Thus, in a further aspect of the invention, the hot melt extruded product is prepared in a manner that substantially excludes air during the extrusion phase of the process. This may be accomplished, for example, by using a Leistritz extruder having a vacuum attachment.

Thus, in some embodiments, the hot melt extruded first component is prepared using a twin screw extruder. A premix of the opioid or opioid complex with an optional opioid antagonist, stabilized PEO polymer, additional excipients, such as an additional nonionic polymer, plasticizer, disintegrants, or lubricants, or combinations thereof, can be introduced into one or two rotating screws that convey the mixture into a heated zone where shear forces are imparted into the mixture, compounding the mixture until a molten mass is achieved. The feed materials enter through a feed hopper and are moved through the barrel by the screws, and are forced through the die into strands that are thereafter conveyed (such as by a continuous movable belt) to allow for cooling and being directed to a pelletizer or other suitable device to render the extruded ropes into the multiparticulate system.

The system can additionally include a temperature control console that includes temperature sensors, cooling means, and temperature indicators throughout the length of the extruder. The pelletizer can consist of rollers, fixed knife, rotating cutter, and the like.

The operating temperature in the extruder is generally in the range of from about 60° C. to about 160° C. Exemplary temperatures for the extruder are determined by those of skill in the art, in accordance with the types of PEO used, for example, about 90° C., about 110° C., about 130° C., or about 150° C., and ranges between any two of these values (including endpoints). Different zones of the extruder may be maintained at different temperatures, as determined by one of skill in the art.

### Preparation of the Second Component

The second component can be prepared by mixing or blending at least one PEO polymer and a stabilizing agent with an optional solvent such as ethanol and optionally adding a complexing agent to the PEO polymer while mixing. The composition can be mixed in a suitable blender, and can be optionally sieved to remove any aggregates or lumps in the material. The mixing time may vary depending on the various components present and the scale of the preparation. In some embodiments, the mixing time is about 5 minutes to about 1 hour, for example, about 5 minutes, about 10 minutes, about 15 minutes, or about 30 minutes.

### Blending of the First and Second Components

The hot melt extruded and milled first component, and the second component, may be mixed in a suitable mixing apparatus, such as a blender, e.g., a V-blender, for an amount of time sufficient to provide the desired product. The mixing time may vary depending on the various components present and the scale of the preparation. In some embodiments, the mixing time is sufficient to provide a substantially homogenous mixture, for example about 5 minutes to about 1 hour, e.g., about 5 minutes, about 10 minutes, about 15 minutes, or about 30 minutes. Optionally, additional excipients can be included in the mixed composition, such as plasticizers, disintegrants, lubricants, or combinations thereof.

### Compression

The mixed composition can be compressed into tablets using a conventional tablet press or by methods commonly known to persons of skill in the art.

### Tablet Curing

Various embodiments of the present technology include at least one curing step wherein the pharmaceutical composition is subjected to an elevated temperature for a specified period of time. In such embodiments, the temperature employed in the curing process is at least as high as the melting temperature of at least one of the PEO polymers contained in the pharmaceutical composition, for example, at least as high as that of the lower weight PEO polymers contained in the pharmaceutical composition. In one embodiment, the temperature employed in the curing process is at least as high as the melting point of the low molecular weight PEO polymer present in the milled hot melt extrudate of the first component. Without being bound to any theory, it is believed that during the curing process, the polymer particles in the second component will diffuse and fuse with the softening milled hot melt extrudate of the first component.

In some embodiments, the solid pharmaceutical composition can be cured by elevated temperatures over a period of time, e.g., cured for about 11 to 24 hours. In some embodiments, the solid pharmaceutical composition is cured for about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours, and ranges between any two of these values (including endpoints). According to some embodiments, the curing temperature is at least about 65° C., at least about 70° C., at least about 75° C., at least about 80° C., at least about 85° C., at least about 90° C., at least about 95° C., or at least about 100° C. According to some embodiments, the curing temperature ranges from about 65° C. to about 100° C., from about 65° C. to about 90° C., from about 70° C. to about 90° C., from about 70° C. to about 85° C., or from about 70° C. to about 80° C. According to some embodiments, the curing temperature is about 70° C., about 71° C., about 72° C., about 73° C., about 74° C., about 75° C., about 76° C., about 77° C., about 78° C., about 79° C., or about 80° C., and ranges between any two of these values (including endpoints).

In some embodiments, the solid pharmaceutical composition can be cured on a tray dryer. The portions of the solid pharmaceutical composition (e.g., tablets) are spread on a tier of perforated trays in a loading chamber. The trays are introduced into a curing device, e.g., convection oven or static oven, and subjected to elevated temperatures, as disclosed above, e.g., heating in a convection oven or a static oven, for example, at a temperature between 65-80° C. for a period of about 11-24 hours.

### 3. Methods of Treatment

In some embodiments, the present invention is directed to a method of treatment wherein a dosage form according to the invention comprising an opioid analgesic is administered for treatment of pain to a patient in need thereof.

In some embodiments, the present invention is directed to a method of treatment wherein a dosage form according to the invention comprising an opioid analgesic (e.g., an extended release, tamper-resistant, abuse-deterrent tablet composition suitable for once or twice daily administration comprising a therapeutically effective amount of oxycodone or a pharmaceutically acceptable salt thereof) is administered for treatment of pain to a patient in need thereof.

In some embodiments, the present invention is directed to the use of a dosage form according to the invention comprising an opioid analgesic for the manufacture of a medicament for the treatment of pain.

In some embodiments the invention is directed to a method of treatment wherein a dosage form is administered for treatment of a disease or certain condition (such as pain) of a patient that requires treatment, and the use of a dosage form according to the invention for the manufacture of a medicament for the treatment of a disease or certain condition (such as pain) of a patient that requires treatment.

In some embodiments the invention is directed to a method of treatment wherein a dosage form is administered for treatment of a disease or certain condition of pediatric patient population that requires treatment, in particular pain, and the use of a dosage form according to the invention for the manufacture of a medicament for the treatment of a disease or certain condition of the pediatric patient population that requires treatment, in particular pain.

### 4. Abuse Deterrence

In some embodiments, the present technology provides a method of deterring abuse of an opioid, comprising:

providing a solid pharmaceutical formulation to a subject in need thereof, wherein the solid pharmaceutical formulation comprises a cured blend of a milled, hot melt extruded first component and a second component,

wherein the milled, hot melt extruded first component comprises an opioid or a pharmaceutically acceptable salt thereof, at least one PEO polymer, at least one additional nonionic polymer (optional), and an oxidative stabilizing agent; and the second component comprises at least one PEO polymer and an oxidative stabilizing agent,

wherein the solid pharmaceutical formulation exhibits lower abuse potential compared to a noncured extended release opioid solid pharmaceutical formulation.

In some embodiments, the solid pharmaceutical formulation is an oral tablet. In specific embodiments, the tablet is compressed.

In some embodiments, the present technology provides a method of decreasing the abuse potential of an opioid, the method comprising:

providing an oral tablet to a subject in need thereof, wherein the tablet comprises a cured blend of a milled hot melt extruded first component and a second component;

the first component comprising an opioid or a pharmaceutically acceptable salt thereof, at least one PEO polymer, at least one additional nonionic polymer (optional), and a stabilizing agent, wherein the first component is hot melt extruded;

the second component comprising at least one PEO polymer and a stabilizing agent; and

wherein the tablet provides an extended release of the opioid.

In some embodiments, the present technology provides a method of decreasing the abuse potential of an opioid, the method comprising:

providing an oral tablet to a subject in need thereof, wherein the tablet comprises a cured blend of a milled melt-granulated first component and a second component;

the first component comprising an opioid or a pharmaceutically acceptable salt thereof, at least one PEO polymer, at least one additional nonionic polymer (optional), a stabilizing agent, and a wax, wherein the first component is melt-granulated;

the second component comprising at least one PEO polymer and a stabilizing agent; and

wherein the tablet provides an extended release of the opioid.

In some embodiments, a tablet of the solid pharmaceutical composition of the invention can withstand a crushing force (e.g., a crushing strength) ranging up to 500 N, up to 600 N, up to 700 N, up to 800 N, up to 900 N, or up to 1000 N, i.e., the tablets are deformed on application of a crushing force (e.g., of up to 1000 N) with less than a 10% fraction (w/w) of particles of about 500 microns or less in size separating from the tablet. Crushing tests can be performed by persons of skill in the art. Exemplary methods for testing crushing (e.g., crushing strength) include striking with a hammer, pressing the tablet between two spoons, using pliers, crushing using pestle and mortar, or testing with a compression tester, such as a diametral compression tester commonly known to persons of skill in the art. During the compression or strength testing in a compression tester, the tablet is placed between two jaws (one of which is fixed, and the other is mobile).

In some embodiments, the unequal distribution of opioid seen in the two fractions (i.e., fines fraction (less than about 75 microns) and coarse fraction (between about 125 microns and about 250 microns)) upon grinding of OXYCONTIN® ORT tablets is not seen with the oxycodone tablets of the present technology. As seen in Table I below, the percentage of drug content in the fines fraction of the ground OXYCONTIN® ORT tablets (RLD) (40 mg; see the two “RLD” columns) was twice as high as would be expected/predicted from the composition of the tablet (i.e., the percentage of drug content in the fines fraction of the ground OXYCONTIN® ORT tablets was not close to that predicted from the composition of the tablet; not at or near about 100%, for example not in the range of about 85% to about 115%, not in the range of about 90% to about 110%; not in the range of about 95% to about 105%). A similar departure from the percentage of drug content predicted in the coarse fraction of the ground OXYCONTIN® ORT tablets, but in the inverse direction, was demonstrated as well (see the two “RLD” columns of Table I).

In stark contrast, with the oxycodone hydrochloride 40 mg tablets of the present technology (Test Product; see the three “HME” columns in Table I), the percentage of drug content in the fines fraction of the ground tablets is close (i.e., at or near about 100%, for example, in the range of about 85% to about 115%, in the range of about 90% to about 110%; in the range of about 95% to about 105%) to that predicted from the composition of the tablet; this is true as well for coarse fraction of the Test product tablets. This drug segregation, i.e., the higher amounts/percentages of opioid in the fines fraction produced by grinding of OXYCONTIN® ORT tablets, results in enhanced potency of drug insufflation (e.g., nasal insufflation) by an abuser. Thus, the oral tablet of the present technology exhibits resistance to drug segregation, whereas the OXYCONTIN® ORT tablet does not, as shown in Table I. For at least this reason, the oral tablet of the present technology is less prone to being abused by snorting (i.e., nasal insufflation) upon grinding compared with the OXYCONTIN® ORT tablet.

In some additional or alternative embodiments, the oral extended-release tablet of the present technology provides opioid drug substance fully embedded in plasticized PEO, HPMC 200K, and KOLLIDON® SR based matrix, preventing the drug from being segregated out of the matrix when subjected to grinding, as shown in Table I.

TABLE I
Drug Content in Different Particle Size Fractions of
Tablets After Being Subjected to Grinding*

| Particle Size | % Drug Content | | | | | |
|---|---|---|---|---|---|---|
| Band | | | Wet | HME** | HME** | HME** |
| (microns) | RLD | RLD | granulation** | (Test) | (Test) | (Test) |
| <75 | 200 | 254 | 161 | 104 | 97 | 95 |
| (“fines | | | | | | |
| fraction”) | | | | | | |
| 125-250 | 65 | 64 | 113 | 98 | 99 | 99 |
| (“coarse | | | | | | |
| fraction”) | | | | | | |

*Grinding conditions: Coffee grinder, Hamilton Beach # 80365. Twenty tablets were ground for a total grinding time of four minutes, following a pattern of one minute grinding/one minute rest.
**All tablets were cured in oven at 75° C. for 24 hours.

In some embodiments, the oral extended-release tablet of the present technology is resistant to dose dumping or extraction with solvents such as ethanol or acetone. In some embodiments, the oral tablet of the present technology provides an in vitro dissolution rate, when measured in a USP Apparatus I (basket) at 100 rpm in 900 ml simulated gastric fluid (SGF) without enzymes, comprising 40% v/v of ethanol at 37° C., characterized by the percent amount of active agent released at about 0.5 hours; or at about 0.5 hours and about 0.75 hours; or at about 0.5 hours, about 0.75, and about 1 hour; or at about 0.5 hours, about 0.75, about 1 hour, and about 1.5 hours; or at about 0.5 hours, about 0.75, about 1 hour, about 1.5, and about 2 hours of dissolution that deviates no more than 20%, or no more than 15%, or no more than 10%, or no more than 5% from the corresponding in vitro dissolution rate measured in a USP Apparatus I (basket) at 100 rpm in 900 ml simulated gastric fluid (SGF) at 37° C. of the reference tablet (ORT).

In some additional embodiments, the in vitro dissolution of dosage form, characterized by the percent amount of active released at 30 minutes of dissolution, when measured in a USP Apparatus I (basket) at 100 rpm in 900 ml simulated gastric fluid comprising 40% v/v of ethanol at 37° C., deviates no more than 20%, or no more than 15%, or no more than 10%, or no more than 5% from the corresponding in vitro dissolution measured in a USP Apparatus I (basket) at 100 rpm in 900 ml simulated gastric fluid at 37° C. without ethanol.

In some additional or alternative embodiments, the oral tablet of the present technology maintains an extended release of the opioid after being subjected to heat treatment, commonly referred to as “crisping” by drug abusers. The heating (e.g., heat pretreatment) may be carried out in a microwave or convection oven, or using a hot plate, lighter, or candle, or the like. For example, in some embodiments, the oral tablet of the present technology maintains an extended release of the opioid after heat pretreatment at about 100° C. for at least about 2 hours in an oven (e.g., a convection oven). In some embodiments, the oral tablet of the present technology maintains an extended release of the opioid after being heat pretreatment in the center of a preheated microwave (e.g., preheated to 80° C.) for about 11-25 minutes (e.g., about 13-16 minutes (e.g., about 14 minutes)) at, e.g., 1200 W power, or 2000 W power. In some embodiments, the tablet of the present technology maintains an extended release of oxycodone after being subjected to heating (e.g., crisping; heating at about 100° C. for at least about 2 hours in an oven; heating in a microwave at 1200 W for about 14 minutes) comparable to, or with a deviation of no more than about 20% from, an extended release oxycodone oral tablet that is not subjected to such heat pretreatment. In some embodiments, the deviation may be no more than about 15%, about 10%, or about 5% from an extended release oxycodone oral tablet that is not subjected to such heat pretreatment.

In some additional or alternative embodiments, the oral tablet of the present technology resists being drawn into a syringe upon extraction with an aqueous solvent. In some embodiments, the oral tablet of the present technology was ground and subject to extraction with about 2 ml to about 10 ml of an aqueous liquid, and the liquid phase was attempted to be withdrawn into a syringe with an 18 or 27 gauge needle and subjected to drug content analysis In another embodiment, the amount of opioid extracted from an oral tablet of the present technology after being ground, crushed, or broken into smaller pieces and subjected to extraction, with about 2 ml to about 10 ml of an aqueous liquid, is less than about 20%, less than about 15%, less than about 10%, less than about 5%, or less than about 3% of the active agent (e.g., oxycodone hydrochloride) in the original tablet.

### 5. Extended Release Profile (In Vitro and In Vivo Testing)

In some embodiments, the oral tablet of the present technology provides a dissolution rate that, when measured in a USP Apparatus I (basket) at 100 rpm in 900 ml aqueous media (e.g., simulated gastric fluid without enzymes (SGF)) at 37° C., is between about 12.5% and about 55% (by wt.) active agent released after 1 hour, between about 25% and about 65% (by wt.) active agent released after 2 hours, between about 45% and about 85% (by wt.) active agent released after 4 hours, between about 55% and about 95% (by wt.) active agent released after 6 hours, and optionally between about 75% and about 100% (by wt.) active agent released after 8 hours. In some embodiments, the oral tablet provides a dissolution rate that, when measured in a USP Apparatus I (basket) at 100 rpm in 900 ml simulated gastric fluid without enzymes (SGF) at 37° C., is between about 15% and about 45% (by wt.) active released after 1 hour, between about 30% and about 60% (by wt.) active agent released after 2 hours, between about 50% and about 80% (by wt.) active agent released after 4 hours, between about 60% and about 90% (by wt.) active agent released after 6 hours, and optionally between about 80% and about 100% (by wt.) active agent released after 8 hours. In particular embodiments, the oral tablet provides a dissolution rate that, when measured in a USP Apparatus I (basket) at 100 rpm in 900 ml SGF without enzymes at 37° C., is between about 17.5% and about 35% (by wt.) active agent released after 1 hour, between about 35% and about 55% (by wt.) active agent released after 2 hours, between about 55% and about 75% (by wt.) active agent released after 4 hours, between about 65% and about 85% (by wt.) active agent released after 6 hours, and optionally between about 85% and about 100% (by wt.) active agent released after 8 hours.

In some embodiments, the present technology provides a twice-a-day oral extended release solid pharmaceutical formulation that provides a mean tₘₐₓ at about 2 hours to about 6 hours, at about 2.5 hours to about 5.5 hours, or at about 2.5 hours to about 5 hours after administration at steady state or of a single dose to human subjects. The solid pharmaceutical formulation is a tablet, which comprises oxycodone or a pharmaceutically acceptable salt thereof.

In some embodiments, the present technology provides a once-a-day solid oral extended release pharmaceutical formulation that provides a mean tₘₐₓ at about 3 hours to about 10 hours, at about 4 hours to about 9 hours, or at about 5 hours to about 8 hours after administration at steady state or of a single dose to human subjects. The dosage form may comprise oxycodone or a pharmaceutically acceptable salt thereof.

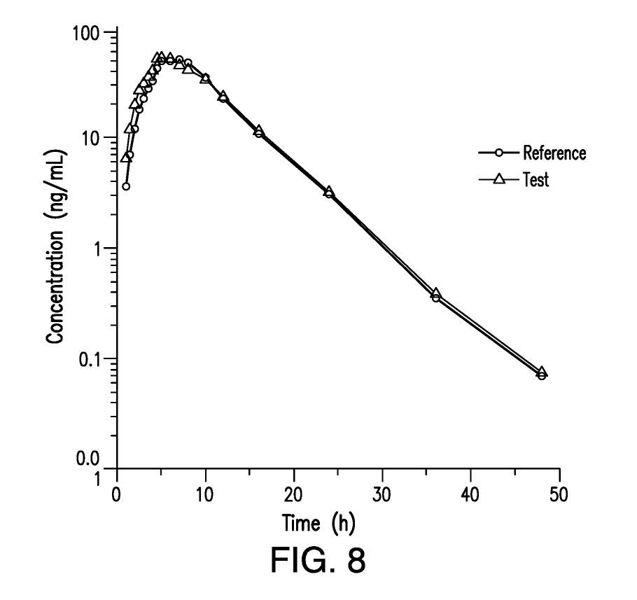

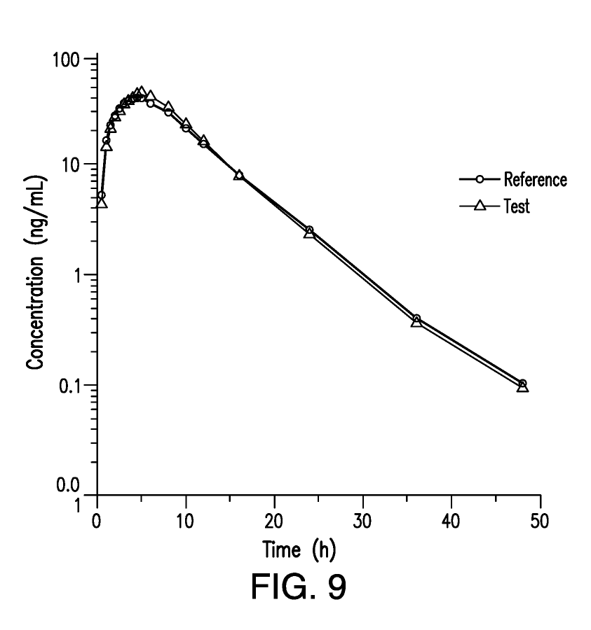

In a further aspect of the invention, a solid oral extended release pharmaceutical formulation of oxycodone hydrochloride is provided that is bioequivalent to the commercial OXYCONTIN® ORT tablet under both fasted and fed conditions. FIGS. 8 and 9 demonstrate that oxycodone hydrochloride extended release tablets (40 mg) according to an embodiment of the present technology are bioequivalent to the commercial OXYCONTIN® ORT tablets (40 mg). FIG. 8 compares human plasma concentration profiles of oxycodone hydrochloride ER tablets (40 mg) of the present technology with OXYCONTIN® ORT tablets (40 mg) under fed conditions. Table II provides human pharmacokinetic parameters of oxycodone and statistical data analyses when oxycodone hydrochloride ER tablets (40 mg) of the present technology were administered under fed conditions.

TABLE II

| | INTRA- | | | | 90% | |
|---|---|---|---|---|---|---|
| | SUBJECT | GEOMETRIC | | | CONFIDENCE | |
| PARAMETER | C.V. (%) | LSMEANS | | RATIO (%) | LIMITS (%) | |
| Cₘₐₓ (ng/mL) | 16.0 | 57.33 | 52.73 | 108.73 | 99.90 | 118.35 |
| AUC_{0-T} (ng · h/mL) | 6.6 | 530.00 | 493.92 | 107.31 | 103.58 | 111.17 |
| AUC_{0-∞}(ng · h/mL) | 6.8 | 540.37 | 503.60 | 107.30 | 103.50 | 111.24 |

FIG. 9 compares human plasma concentration profiles of oxycodone hydrochloride ER tablets of the present technology with OXYCONTIN® ORT tablets (40 mg) under fasting conditions.

Table III provides human pharmacokinetic parameters of oxycodone and statistical data analyses when oxycodone hydrochloride ER tablets (40 mg) of the present technology were administered under fasting conditions.

TABLE III

| | INTRA- | | | | 90% | |
|---|---|---|---|---|---|---|
| | SUBJECT | GEOMETRIC | | | CONFIDENCE | |
| PARAMETER | C.V. (%) | LSMEANS | | RATIO (%) | LIMITS (%) | |
| Cₘₐₓ (ng/mL) | 13.4 | 46.140 | 40.596 | 113.66 | 107.29 | 120.41 |
| AUC_{0-T} (ng · h/mL) | 11.3 | 442.594 | 418.212 | 105.83 | 111.09 | 111.09 |
| AUC_{0-∞} (ng · h/mL) | 11.1 | 446.169 | 442.307 | 105.65 | 100.70 | 110.85 |

### EXAMPLES

This disclosure is further illustrated by the following examples, which are not to be construed as limiting this disclosure in scope or spirit to the specific procedures described herein. It is to be understood that the examples are provided to illustrate certain embodiments and that no limitation to the scope of the disclosure is intended. It is to be further understood that various other embodiments, modifications, and equivalents thereof which may suggest themselves to those skilled in the art without departing from the spirit of the present disclosure and/or scope of the claims.

### Examples 1-6

Exemplary Formulations and Methods of Making

TABLE IV
Exemplary Formulations of Extended Release Oral Tablets (Examples 1-6)

| | | | | | | |
|---|---|---|---|---|---|---|
| Component | Example 1 | Example 2 | Example 3 | Example 4 | Example 5 | Example 6 |

First component (mg/tablet)

| | | | | | | |
|---|---|---|---|---|---|---|
| Oxycodone HCl | 40.0 | 40.0 | 40.0 | 40.0 | 40.0 | 40.0 |
| DL-α-tocopherol | 0.45 | 0.45 | 0.45 | 0.45 | 0.45 | 0.45 |
| POLYOX ® WSR | 63.775 | — | 46.025 | — | — | — |
| N-301 LEO | | | | | | |
| POLYOX ® WSR | — | 63.775 | — | 55.275 | 56.025 | 56.025 |
| N-1105 LEO | | | | | | |
| HPMC-K200M | — | — | 20.0 | — | 5.0 | 5.0 |
| Kollidon ® SR | — | — | 7.5 | 15.0 | 7.5 | 7.5 |
| Triethyl citrate | — | — | 8.0 | 2.0 | 3.0 | 3.0 |

Second component (mg/tablet)

| | | | | | | |
|---|---|---|---|---|---|---|
| POLYOX ® WSR | — | — | 46.025 | — | — | 56.025 |
| N-301 LEO | | | | | | |
| POLYOX ® WSR | 63.775 | 63.775 | — | 55.275 | 56.025 | — |
| N-1105 LEO | | | | | | |
| DL-α-tocopherol | 0.45 | 0.45 | 0.45 | 0.45 | 0.45 | 0.45 |

Final Blend (mg/tablet)

| | | | | | | |
|---|---|---|---|---|---|---|
| Magnesium | 2.0 | 2.0 | 2.0 | 2.0 | 2.0 | 2.0 |
| stearate | | | | | | |
| Total tablet | 170.45 | 170.45 | 170.45 | 170.45 | 170.45 | 170.45 |
| weight (mg) | | | | | | |

Exemplary formulations presented in Table IV were prepared as follows:

Oxycodone hydrochloride (40 mg) and the PEO polymer were dry mixed in a high shear mixer with the impeller set at low speed to create a premix blend. A solution containing dl-alpha-tocopherol dissolved in ethanol was sprayed onto the premix blend, creating the first component. The first component was extruded through a hot melt extruder (Leistritz Hot Melt Extruder Micro-18). The extruder had a constant feed rate of 15 g/min and a screw speed at 100 rpm. The temperature zones were kept at 90° C. (zone 1), 110° C. (zone 2), 110° C. (zone 3), 110° C. (zone 4), and 110° C. (die). The extrudate was cooled and cut into strands. The extruded strands were milled (Fitz mill with 3.18 mm pore size) at a high speed. The resulting particles were further reduced by milling (Fitz mill with 0.844 mm pore size).

To produce the second component, the PEO polymer was uniformly blended with dl-alpha-tocopherol in an ethanolic solution. The blend was screened through a standard #20 mesh sieve.

The first component and the second component were blended in a V-blender for 5 minutes at 25 rpm. Prescreened magnesium stearate (standard #30 mesh sieve) was added and blended for 3 minutes at 25 rpm. The resultant mixture was the blended composition.

The blended composition was compressed into matrix tablets with round biconvex shape using a “B Tool” rotary tablet press. These matrix tablets were cured under static or dynamic conditions by a tray dryer. The core tablets were spread on tiers of perforated trays in a loading chamber with forced circulation of 300 CFM of heated air at 75° C. for 11 to 24 hours.

The tablets shown in Examples 2-6, as shown in Table IV above, were prepared according to procedures similar to that of Example 1. In Examples 3, and 5-6, HPMC was added to the premix blend creating the first component. In Examples 3-6, Kollidon was added to the premix blend along with HPMC, when present, creating the first component. In Examples 3-6, which include a plasticizer (triethyl citrate), the plasticizer was added to the solution containing DL-α-tocopherol prior to spraying onto the premix blend of the first component.

### Example 7

TABLE V
Exemplary Formulation of Extended Release
Oral Tablets with Opioid Antagonist
Formulation composition for Oxycodone HCl
tablets containing Naloxone hydrochloride

| | mg/Tablet |
|---|---|
| First component | |
| Oxycodone hydrochloride | 40.0 |
| Colloidal silicon dioxide | 40.0 |
| Naloxone HCl | 20.0 |
| DL-α-tocopherol | 0.40 |
| POLYOX ® WSR N-1105 LEO | 100.0 |
| HPMC-K200M | 5.0 |
| Kollidon ® SR | 7.5 |
| Triethyl citrate | 3.0 |
| Second component | |
| DL-α-tocopherol | 0.40 |
| POLYOX ® WSR N-301 LEO | 159.7 |
| POLYOX ® WSR N-303 LEO | — |
| Final blend | |
| Magnesium stearate | 4.0 |
| Total tablet weight (mg) | 380 |

An exemplary formulation, as presented in Table V, is prepared as follows:

Aqueous oxycodone hydrochloride solution is mixed with colloidal silicon dioxide in a high shear mixer to uniformly entrap the drug solution into pores of the colloidal silica. The resulting mixture is milled and mixed to obtain uniform 80 mg drug particles (equivalent to 40 mg of oxycodone hydrochloride).

A solution containing dl-alpha-tocopherol and triethyl citrate is sprayed onto the premix blend. Additional agents, e.g., HPMC, Kollidon SR, and naloxone hydrochloride, are mixed into the premix blend. The resulting blend creates the first component. The first component is extruded through a melt extruder (Leistritz Hot Melt Extruder Micro-18). The extruder has a constant feed rate of 15 g/min and a screw speed at 100 rpm. The temperature zones are kept at 90° C. (zone 1), 110° C. (zone 2), 110° C. (zone 3), 110° C. (zone 4), and 110° C. (die). The extrudate is cooled and cut into strands. The extruded strands are milled (Fitz mill with 3.18 mm pore size) at a high speed. The resulting particles are further reduced by milling (Fitz mill with 0.844 mm pore size).

To produce the second component, the PEO polymer is uniformly blended with dl-alpha-tocopherol in an ethanolic solution. The blend is screened through a standard #20 mesh sieve.

The first component and the second component are blended in a V-blender for 5 minutes at 25 rpm. Prescreened magnesium stearate (standard #30 mesh sieve) is added and blended for 3 minutes at 25 rpm.

The blended composition is compressed into matrix tablets with round biconvex shape using a “B Tool” rotary tablet press. These matrix tablets are cured under static or dynamic conditions by a tray dryer. The core tablets are spread on tiers of perforated trays in a loading chamber with forced circulation of 300 CFM of heated air at 75° C. for about 11 hours to about 24 hours.

### Example 8

TABLE VI
Exemplary Formulation of Extended Release
Oxycodone Abuse-Deterrent Tablets
Formulation composition for 40 mg Oxycodone HCl

| Ingredients | mg/Tablet |
|---|---|
| Oxycodone HCl | 40 |
| Kollidon SR | 2 |
| Syloid (244FP) | 3.4 |
| Magnesium stearate | 2.04 |
| Blend 1 | |
| Polyox WSR-1105 (mw 900,000 Da) + Vitamin E | 48.215 |
| Blend 2 | |
| Polyox WSR-1105 (mw 900,000 Da) + Vitamin E | 14.565 |
| Polyox WSR-303 (mw 7,000,000 Da) + Vitamin E | 62.78 |
| Blend 3 | |
| Hypromellose (HPMC K200M), Benecel | 4 |
| Triethyl citrate, NF | 3 |
| Total core weight | 180 |
| Seal coat, Opadry II Clear | 4 |
| Color Coat, Opadry II Yellow | 6 |
| Total tablet weight | 190 |

The exemplary formulation presented in Table VI was prepared as follows:

Intermediate Blend 1: The appropriate amount of Polyox WSR 1105-LEO was weighed and mixed with dl-alpha-tocopherol dissolved in 0.45% w/w of 200 proof ethanol in a stainless steel container in a high shear mixer (Mixer: low/chopper: off) for 5 minutes to provide the intermediate blend of Polyethylene Oxide, NF.

Intermediate Blend 2: The required amounts of Polyox WSR 303-LEO and Polyox WSR 1105-LEO were weighed and mixed with dl-alpha-tocopherol dissolved in 0.45% w/w of ethanol (200 proof) in a stainless steel container in a high shear mixer (Mixer: low/chopper: off) for 5 minutes to provide the intermediate blend 2 of Polyethylene Oxide, NF.

Intermediate Blend 3: The required amount of Hypromellose, NF (Benecel K200M) was weighed and mixed with triethyl citrate dissolved in 0.45% w/w of ethanol (200 proof) in a stainless steel container in a high shear mixer (Mixer: low/chopper: off) for 5 minutes to provide the intermediate blend 3 of Hypromellose, NF (Benecel K200M)—Triethyl Citrate, NF.

Process for manufacture of Formulation: The required amount of Intermediate 1 and 3 were weighed and hand sieved through a #20 sieve. The sieved material was added to a high shear mixer bowl. Oxycodone HCl, KOLLIDON SR and colloidal silicon dioxide (50% of the total amount of silicon dioxide used) are weighed and sieved through mesh #20 and added to the bowl. Mixing was carried out for 5 minutes (Mixer: low and Chopper: off). The discharged material was hot melt extruded (Conditions: Zones 1-4: 110° C., feed rate: 40 g/min). The extruded material was milled (Two stage milling, #1532-0125 and #1532-0040).

The milled material was final blended with Intermediate 2, magnesium stearate, and the remaining amount of silicon dioxide; the resulting blend was compressed into tablets, and cured at 75° C. for 16 hours. The cured tablets were seal coated, and further color coated to manufacture the final product.

Additional formulation compositions for oxycodone extended release tablets are shown in Tables VII and VIII below.

TABLE VII

| Ingredients | 40 mg | 40 mg | 10 mg |
|---|---|---|---|
| Oxycodone HCl, USP (Fine Grade) | 40 | 40 | 10 |
| Hypromellose, NF (Benecel | 7 | 7 | 7 |
| K200M Pharm) | | | |
| KOLLIDON SR | 3.5 | 3.5 | 3.5 |
| Vitamin E, USP (DL-α- | 0.47 | 0.44 | 0.51 |
| Tocopherol) | | | |
| Triethyl citrate, NF | 3 | 3 | 3 |
| PEO, NF (Sentry Polyox | 57.423 | 57.46 | 72.425 |
| WSR 1105-LEO) | | | |
| PEO, NF (Sentry Polyox | 57.467 | 57.46 | 72.425 |
| WSR 303) | | | |
| Colloidal Silicon Dioxide, NF | 3.4 | 3.4 | 3.4 |
| (Syloid) | | | |
| Magnesium stearate, NF | 1.74 | 1.74 | 1.74 |
| Seal Coating: Opadry II clear | 4 | — | 4 |
| Color coating: Opadry based* | 6 | 12 | 6 |
| Total | 184 | 186 | 184 |

TABLE VIII

| Ingredients | 10 mg | 40 mg | 80 mg |
|---|---|---|---|
| Oxycodone HCl | 10 | 40 | 80 |
| Hypromellose (HPMC | 4 | 4 | 6 |
| K200M), Benecel | | | |
| Triethyl citrate, NF | 3 | 3 | 4.5 |
| Kollidon ® SR | 2 | 2 | 3 |
| Polyox WSR-1105 (mw | 12.053 | 48.215 | 96.424 |
| 900,000 Da) + Vitamin E | | | |
| Syloid (244FP) | 1.7 | 1.7 | 2.55 |
| Polyox WSR-303 (mw | 65.727 | 14.565 | 9.746 |
| 7,000,000 Da) + Vitamin E | | | |
| Polyox WSR-1105 (mw | 77.78 | 62.78 | 106.17 |
| 900,000 Da) + Vitamin E | | | |
| Syloid (244FP) | 1.7 | 1.7 | 2.55 |
| Magnesium stearate | 2.04 | 2.04 | 3.06 |
| Total, mg | 180 | 180 | 314 |

Similar to the oxycodone formulations noted above, other opioids such as oxymorphone, hydrocodone, tapentadol, and hydromorphone can be formulated into solid pharmaceutical formulations of the present technology.

Exemplary formulations for extended release oxymorphone are shown in Table IX below.

TABLE IX

| Ingredients | 40 mg | 40 mg | 10 mg |
|---|---|---|---|
| Oxymorphone HCl, USP (Fine Grade) | 40 | 40 | 10 |
| Hypromellose, NF (Benecel K200M | 7 | 7 | 7 |
| Pharm) | | | |
| KOLLIDON SR | 3.5 | 3.5 | 3.5 |
| Vitamin E, USP (DL-α-Tocopherol) | 0.47 | 0.44 | 0.51 |
| Triethyl citrate, NF | 3 | 3 | 3 |
| PEO, NF (Sentry Polyox WSR 1105- | 57.423 | 57.46 | 72.425 |
| LEO) | | | |
| PEO, NF (Sentry Polyox WSR 303) | 57.467 | 57.46 | 72.425 |
| Colloidal Silicon Dioxide, NF | 3.4 | 3.4 | 3.4 |
| (Syloid) | | | |
| Magnesium stearate, NF | 1.74 | 1.74 | 1.74 |
| Seal Coating: Opadry II clear | 4 | — | 4 |
| Color coating: Opadry based* | 6 | 12 | 6 |
| Total | 184 | 186 | 184 |

### Example 9: Dissolution in Simulated Gastric Fluid (SGF)

The oral tablets of the present technology were tested for their drug release properties by dissolution testing in SGF. Dissolution testing was carried out using the following parameters.

| Dissolution | Simulated Gastric | |
|---|---|---|
| Medium: | Fluid (without enzymes) | |
| Volume: | 900 | mL |
| Temperature: | 37.0° C. ± 0.5° C. | |
| Apparatus: | USP Apparatus I | |
| | (Basket) | |
| Rotation Speed: | 100 | rpm |
| Distance | 2.5 | cm |
| from Bottom: | | |

Analysis was conducted using an HPLC system equipped with a pump, an autosampler, UV or PDA detector, and a suitable data acquisition system using UV at 280 nm. Analytic techniques are commonly known to those of skill in the art.

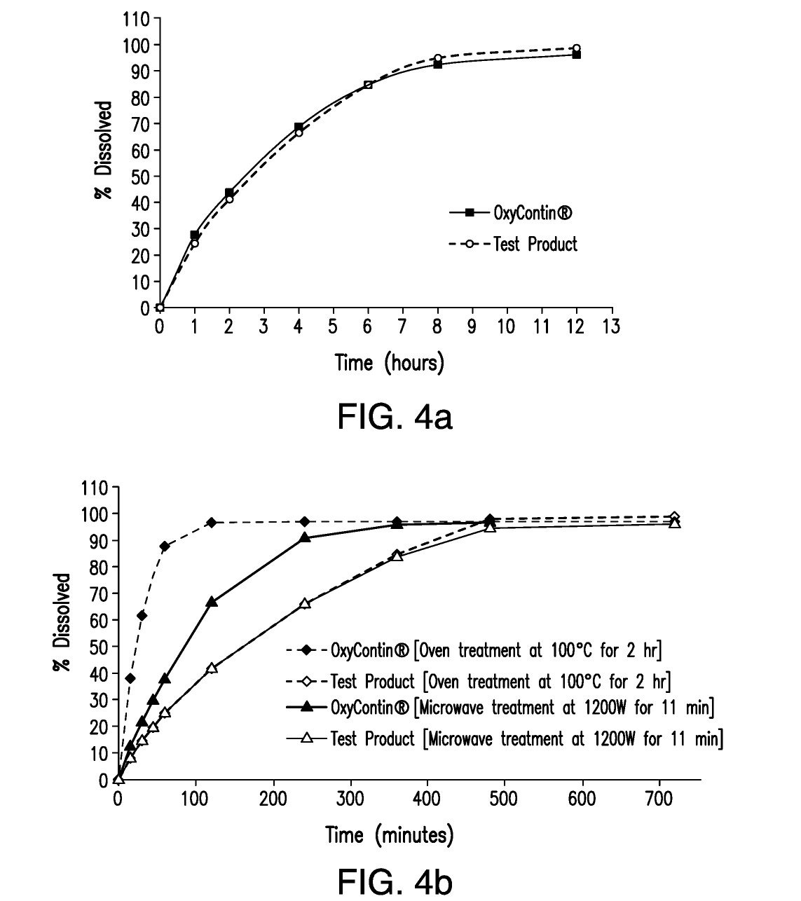

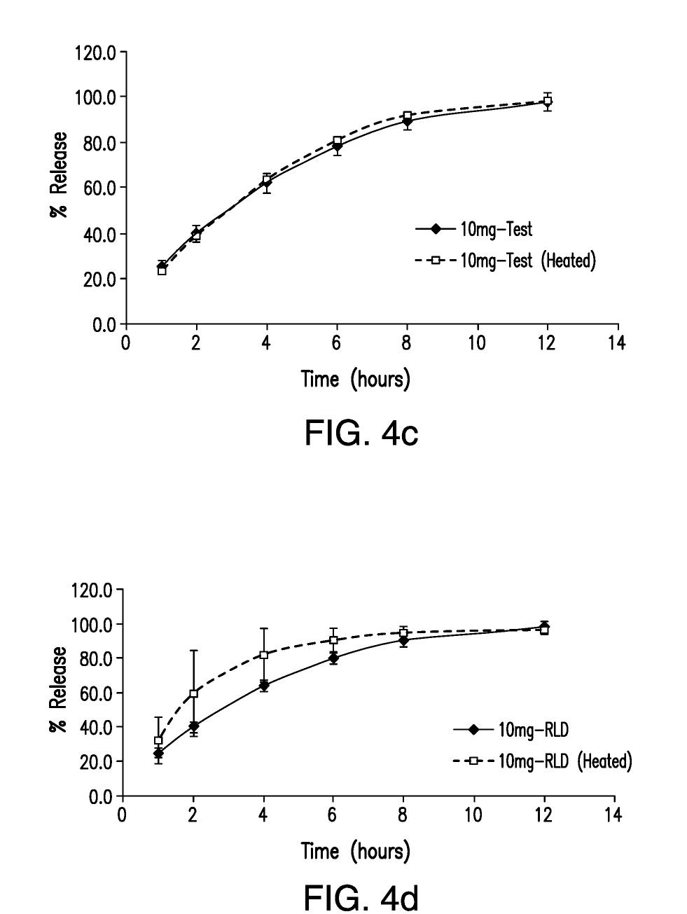

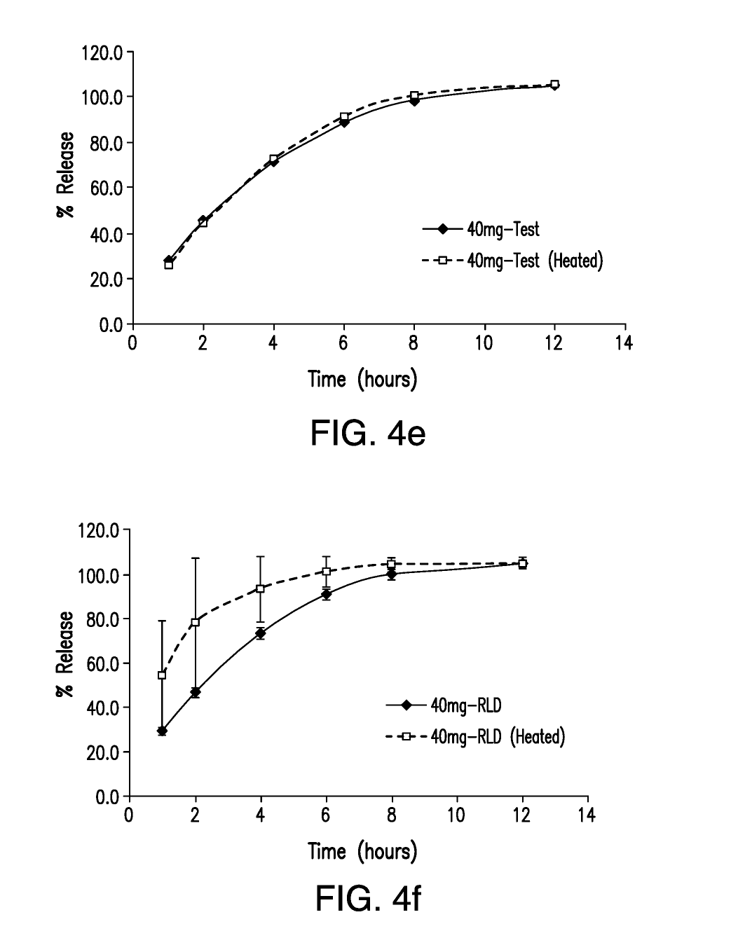

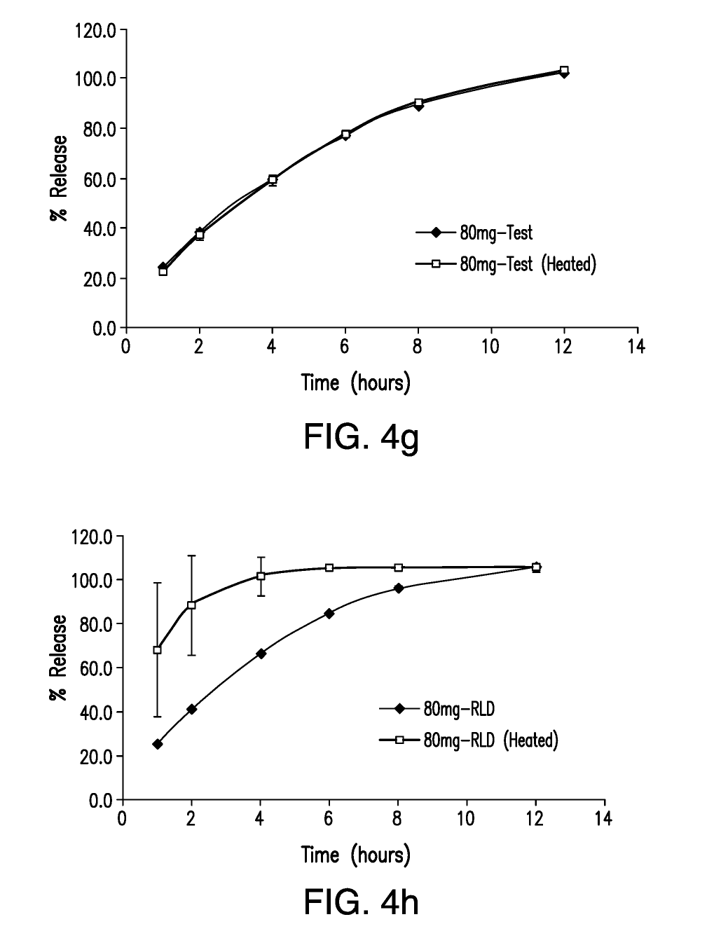

As seen in FIG. 4a, oxycodone oral tablets of the present technology (Test) achieve extended release over a period of up to 12 hours, comparable to OXYCONTIN® ORT tablets (RLD) when not subjected to abuse (e.g., heat pretreatment). As seen in FIG. 4b, the Test product maintains the extended release after heat pretreatments, e.g., oven treatment at 100° C. for two hours or microwave treatment at 1200 W for 11 minutes, whereas the RLD loses its extended release properties on similar heat pretreatments. FIGS. 4c, 4e, and 4g show the dissolution profile in SGF for the Test product (Test) (10, 40, and 80 mg tablets, respectively), with and without heat pretreatment in an oven at 100° C. for two hours. FIGS. 4d, 4f, and 4h show the dissolution profile in SGF for OXYCONTIN® ORT tablets (RLD) (10, 40, 80 mg tablets, respectively), with and without heat pretreatment in an oven at 100° C. for two hours. The figures demonstrate that OXYCONTIN® ORT tablets (10, 40, 80 mg tablets) lose their extended release properties on heating, whereas the Test product maintains its extended release properties for about 12 hours.

### Example 10: Dissolution in 40% v/v Alcohol in Simulated Gastric Fluid

The oral tablets of the present technology were tested for their drug release properties by dissolution testing in 40% v/v ethanol in SGF. Dissolution testing was carried out using the following parameters.

| Dissolution | 40% v/v ethanol in | |
|---|---|---|
| Medium: | SGF (without enzymes) | |
| Volume: | 900 | mL |
| Temperature: | 37.0° C. ± 0.5° C. | |
| Apparatus: | USP Apparatus I | |
| | (Basket) | |
| Rotation Speed: | 100 | rpm |
| Distance | 2.5 | cm |
| from Bottom: | | |

Analysis was conducted using an HPLC system equipped with a pump, an autosampler, UV or PDA detector, and a suitable data acquisition system using UV at 280 nm. Analytic techniques are commonly known to those of skill in the art.

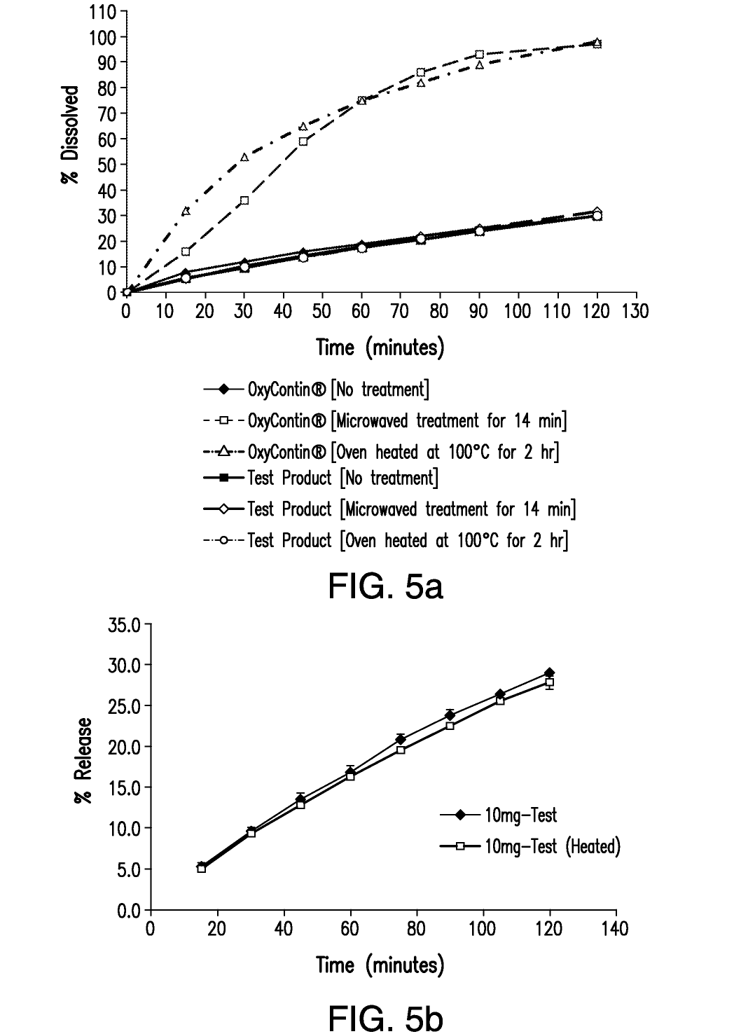

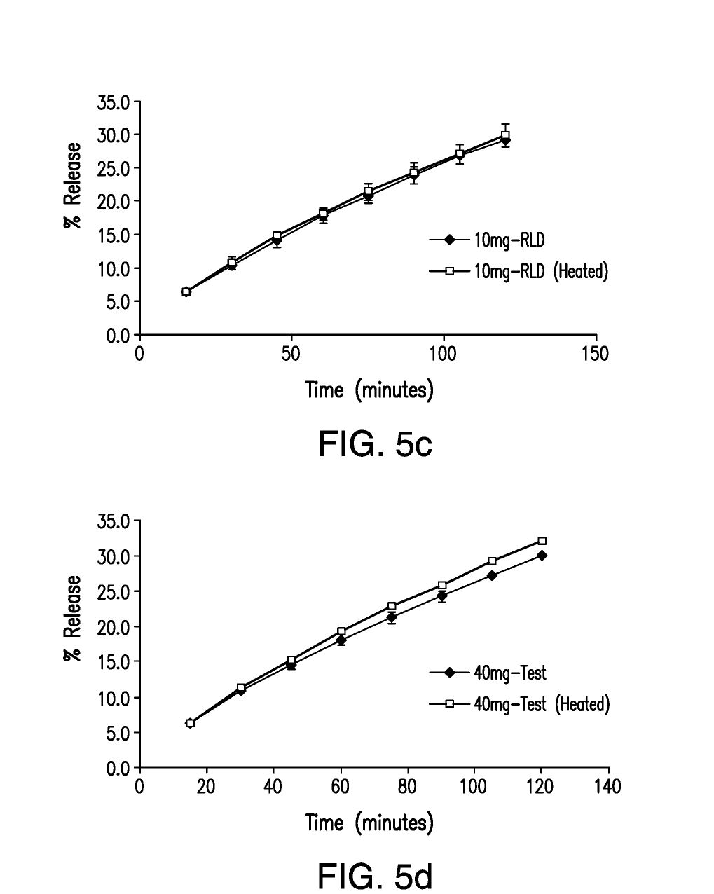

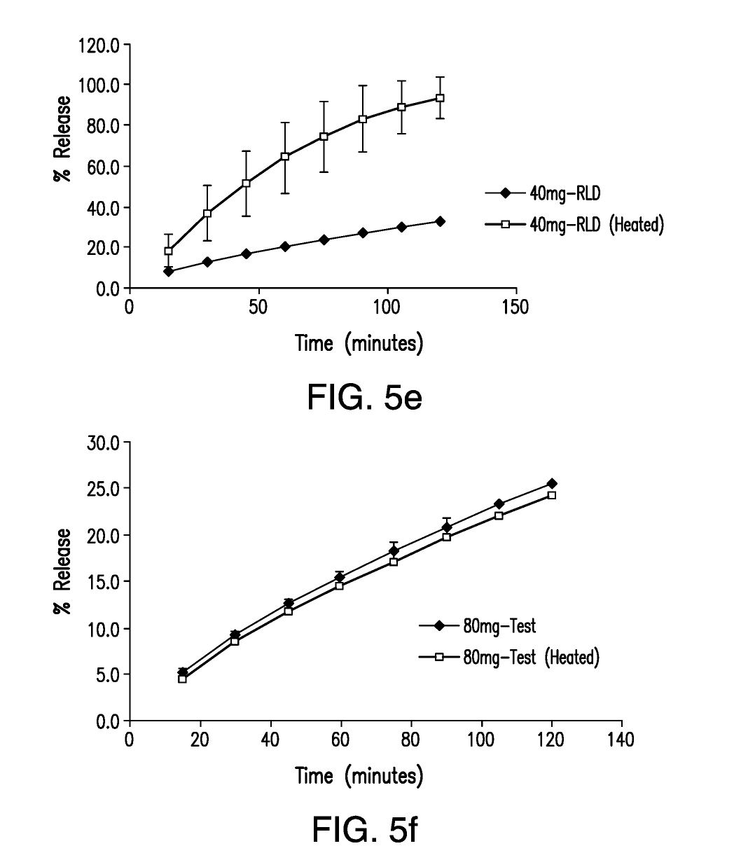

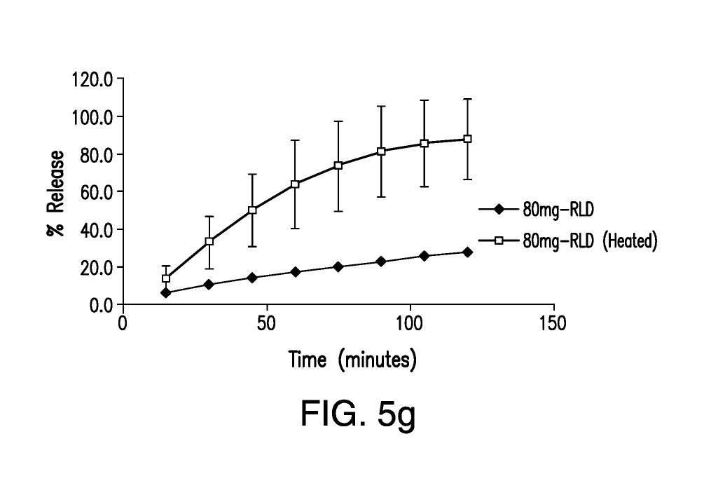

FIG. 5a demonstrates the dissolution properties of oxycodone hydrochloride extended release tablets (40 mg) according to an embodiment of the present technology (Test) compared with OXYCONTIN® ORT tablets (RLD), using 40% alcohol-induced dose dumping dissolution method, before and after either heat pretreatment in an oven at 100° C. for two hours, or heat pretreatment by microwave irradiation at 1200 W for 14 minutes. The Test product maintains the extended release after heat pretreatments, whereas the RLD loses its extended release properties on similar heat pretreatments. FIGS. 5b, 5d, and 5f show the dissolution profile for the Test product (Test), using 40% alcohol-induced dose dumping dissolution method (10 mg, 40 mg, and 80 mg tablets, respectively), with and without heat pretreatment in an oven at 100° C. for two hours. FIGS. 5c, 5e, and 5g show the dissolution profile for OXYCONTIN® ORT tablets (RLD) using 40% alcohol-induced dosing dumping dissolution method (10 mg, 40 mg, 80 mg tablets, respectively), with and without heat pretreatment in an oven at 100° C. for two hours. The figures demonstrate that OXYCONTIN® ORT tablets (40 mg and 80 mg tablets) lose their extended release properties on heating, whereas the Test product maintains its extended release properties for about 12 hours.

### Example 11: Evaluation of Abuse-Deterrent Properties

The tablets of the present technology were evaluated for their abuse-deterrent properties as detailed herein. These tests are meant to simulate the physical manipulations to which an abuser would likely subject the tablets.

Tablet Crush Resistance Testing

Oxycodone extended release tablets according to the present technology (10, 40, and 80 mg tablets) were demonstrated to be crush resistant by several testing methods, including (a) putting the tablet in a plastic bag and striking with a metal hammer 10 times; (b) placing the tablet in a pill crusher and tightening the cap for two minutes; (c) grinding the tablet in a mortar with a pestle for two minutes; and (d) crushing the tablet between two spoons for one or two minutes.

Particulate Evaluation Upon Grinding

Oxycodone extended release tablets according to an embodiment of the present technology were evaluated for the size of the particulate matter upon grinding as follows. The electrical lab grinder (IKA Werke, Model A10 B S2) was assembled with a circulating bath set to 18° C. Tablets were weighed (approximately 5 grams), and ground for a total grinding time of four minutes following a pattern of 2 minutes grinding/1 minute rest/2 minutes grinding. Powder was recovered from the grinder, and the recovery yield was determined and analyzed from the ground powder via sieve analysis using mesh 10, 18, 35, 60, 120 and 230 sieves. The amount of powder retained on each sieve was determined.

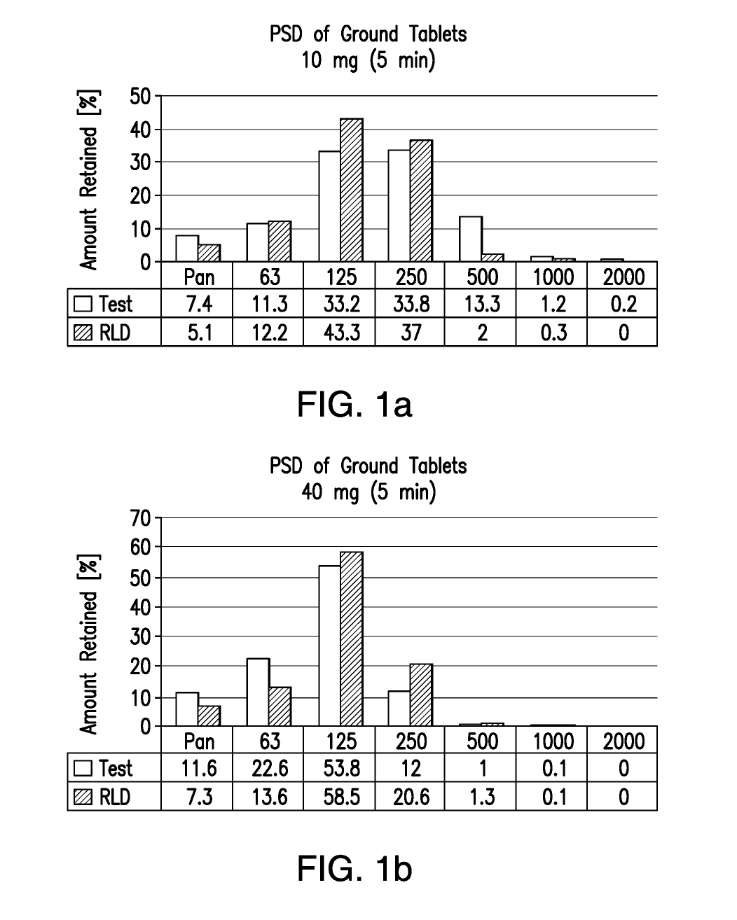

As seen in FIG. 1a, 10 mg oxycodone tablets of the present technology (Test) exhibited particle size distribution of the ground tablets comparable to OXYCONTIN® 10 mg ORT tablets (RLD). Similarly, FIG. 1b demonstrates that 40 mg oxycodone tablets of the present technology (Test) exhibited particle size distribution of the ground tablets comparable to OXYCONTIN® 40 mg ORT tablets (RLD). The size distribution of the particles after grinding generally deters abuse by snorting.

Importantly, and surprisingly, as shown in Table I above, the unequal distribution of opioid seen in two fractions, (1) fines fraction (e.g., less than about 75 microns) and (2) coarse fraction (e.g., about 125 microns to about 250 microns), as determined in a band assay of ground powder, that was seen upon grinding of OXYCONTIN® ORT tablets (RLD), was not seen with the oxycodone tablets of the present technology (Test). According to Table I, the percentage of drug content in the fines fraction of the ground RLD tablets was shown to be about 200-254% of that predicted from the composition of the tablet. Such drug segregation, i.e., the higher amounts/percentages of opioid in the fines fraction produced by grinding of OXYCONTIN® ORT tablets (as compared with the present technology) leads to enhanced potency of drug insufflation (e.g., nasal insufflation) by an abuser of OXYCONTIN® ORT tablets. Thus, the oral tablet of the present technology exhibits resistance to drug segregation, whereas the OXYCONTIN® ORT tablet does not. For at least this reason, the oral tablet of the present technology is less prone to being abused by snorting (i.e., nasal insufflation) upon grinding compared with the OXYCONTIN® ORT tablet.

Assayed API amounts found in each sieve fractions were compared versus the theoretical predicted API amounts calculated from the weight of the powder and the API drug loading of the formulation. First the particle size distribution data in percent are transformed to the predicted amount of API (normalized to one tablet) in each sieve fraction by multiplying the percent retained in each sieve by the product strength and dividing by 100.

The actual amount of API found in each sieve fraction is then compared to the predicted amount. Segregation potential was estimated by calculating the variance of the API amount different from zero, where zero is defined as “no segregation” as the theoretical value and measured value are equal.

Segregation for all strengths of the reference product (OXYCONTIN®) and the Test Product were calculated. The variances of the segregation were calculated according to the following equation:

$$\frac{{\sum }_{1}^{n} {( Δ m - \overline{Δ m} )}^{2}}{n}$$

where Δm is the API difference, Δ̅m̅ is the average off the API difference, and n is the number of sieve fractions. The variance of the segregation is presented in Table X.

TABLE X
Variance of API Segregation across Sieve Fractions

| | | 10 mg | 40 mg | 80 mg |
|---|---|---|---|---|
| Untreated | Test Product | 0.4 | 0.8 | 2.1 |
| | Reference Product | 0.6 | 5.3 | 29.3 |
| | OXYCONTIN ® | | | |
| Heated | Test Product | 0.5 | 0.9 | 1.0 |
| | Reference Product | 0.3 | 5.2 | 24.3 |
| | OXYCONTIN ® | | | |

The variances of API segregation for the 40 mg and 80 mg strengths for the Test Product show a remarkable lack of drug segregation (variances ranging from 0.8 to 2.1), especially in light of the corresponding results for OXYCONTIN® (variances ranging from 5.2 to 29.3). The lack of drug segregation for the Test Product, as compared with OXYCONTIN®, was evident in both untreated tablet and heated tablet dosage forms.

Syringeability

Solution for syringeability study was prepared as follows. A one-tablet equivalent was weighed out into a 20 mL scintillation vial. 2, 5, or 10 mL of water was added to the scintillation vial, vortexed on high for 15 seconds, and allowed to sit without disturbing. After 2 or 10 minutes, the needle of a syringe (18 or 27 gauge needle) was placed into the solution and the liquid was aspired into the syringe for either 60 seconds or until all liquid was drawn up, whichever one occurred first. The amount of oxycodone present in the syringe was quantitated by HPLC analysis.

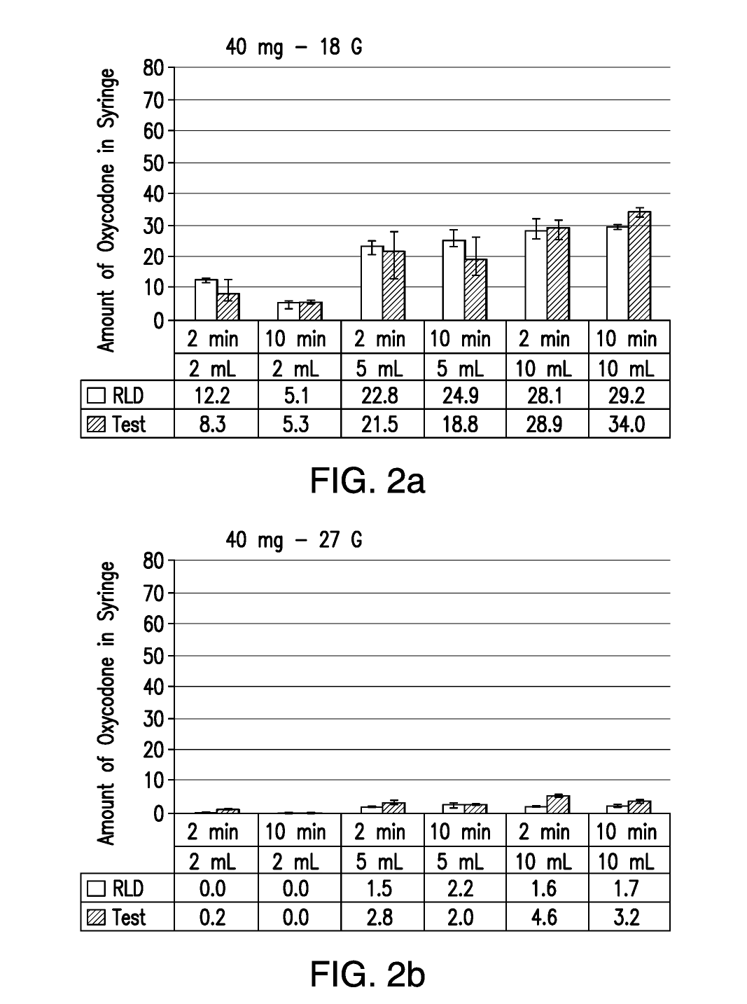

Syringeability results of oxycodone extended release tablets of the present technology (Test) compared to OXYCONTIN® 40 mg ORT tablets (RLD) are provided in FIGS. 2a and 2b, when not subjected to any heat pretreatment. As shown in FIGS. 2a (18 gauge needle) and 2b (27 gauge needle), the results from syringeability studies of oxycodone extended release tablets according to the present technology are comparable to OXYCONTIN® 40 mg ORT tablets when not subjected to any heat pretreatment.

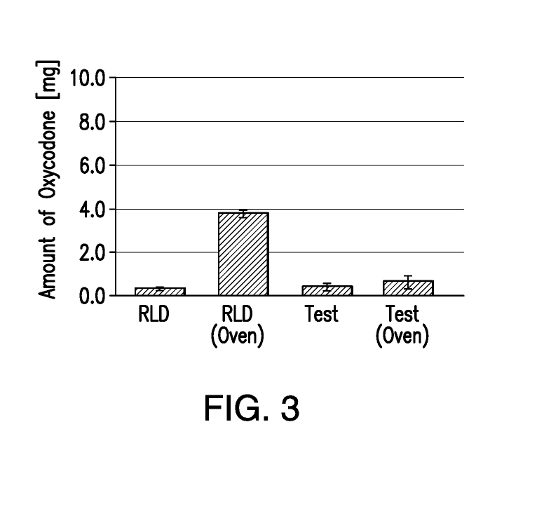

Syringeability results with oxycodone extended release tablets of the present technology (Test) compared to OXYCONTIN® 10 mg tablets (RLD), with and without heat pretreatment in an oven at 100° C. for 2 hours, are provided in FIG. 3. The results indicate that syringeability protection of OXYCONTIN® 10 mg ORT tablets was defeated when subjected to heat pretreatment in an oven for 2 hours. In contrast, oxycodone extended release tablets according to the present technology maintained their syringeability protection property after the same heat pretreatment.

Dose Dumping after Heat Pretreatment

Oven pretreatment—Each tablet (e.g., 40 mg tablet) was placed into a glass vial and placed into an oven preheated to 100° C. The tablet was incubated for two hours. After heat pretreatment, the tablets were tested for dissolution and extended release properties as detailed above.

The microwave oven was preheated to about 80° C. as measured at or near the center of the microwave tray. The tablets were pretreated individually. A single tablet on a watch glass was placed at the center of the microwave tray and microwaved at max power (2000 W) for 14 minutes. If more than one tablet had to be subjected to microwave radiation, the watch glass was removed from the microwave tray in between pretreatment of the tablets and the temperature of microwave oven was allowed to cool down to about 80° C. before performing another tablet pretreatment. The resultant treated tablets were tested for their extended release properties by dissolution testing as detailed above.

FIG. 4a demonstrates that the results from dissolution testing of oxycodone extended release tablets according to the present technology (Test) are comparable to OXYCONTIN® 40 mg ORT tablets (RLD) using the OGD method when not subjected to any heat pretreatment.

FIG. 4b demonstrates the results from dissolution testing of oxycodone extended release tablets according to the present technology (Test) after heat pretreatment by either oven heating or microwave compared to OXYCONTIN® ORT 40 mg tablets, using the OGD method. As seen in FIG. 4b, OXYCONTIN® ORT tablets lose their extended release property after oven heat pretreatment at 100° C. for 2 hours, and begin to lose their extended release properties after microwave heating at 1200 W for 11 minutes. In contrast, oxycodone extended release tablets according to the present technology maintain their extended release properties even after heat pretreatments.

FIG. 5a demonstrates the results from dissolution testing of oxycodone extended release tablets according to the present technology (Test) after heat pretreatment by either oven or microwave heating, as compared to OXYCONTIN® 40 mg ORT tablets in 40% alcohol-induced dose dumping dissolution method (USP Apparatus I (basket) in SGF (without enzymes):ethanol (60:40, v/v); speed=100 rpm; volume=900 ml). As seen in FIG. 5a, OXYCONTIN® ORT tablets begin to lose their alcohol (i.e., ethanol)-induced dose dumping protection property after oven heat pretreatment at 100° C. for 2 hours, as well as after microwave heat pretreatment at 1200 W for 14 minutes. In contrast, oxycodone extended release tablets according to the present technology (Test) maintained their alcohol-induced dose dumping protection property after oven heat pretreatment at 100° C. for 2 hours, as well as after microwave heat pretreatment at 1200 W for 14 minutes.

Extraction Studies of Cut Tablets

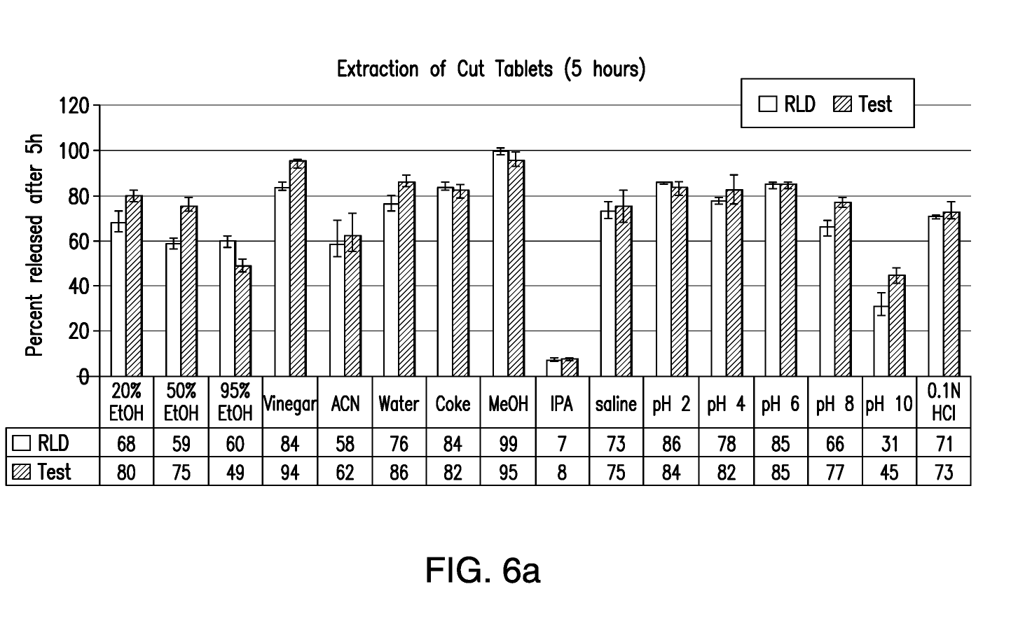

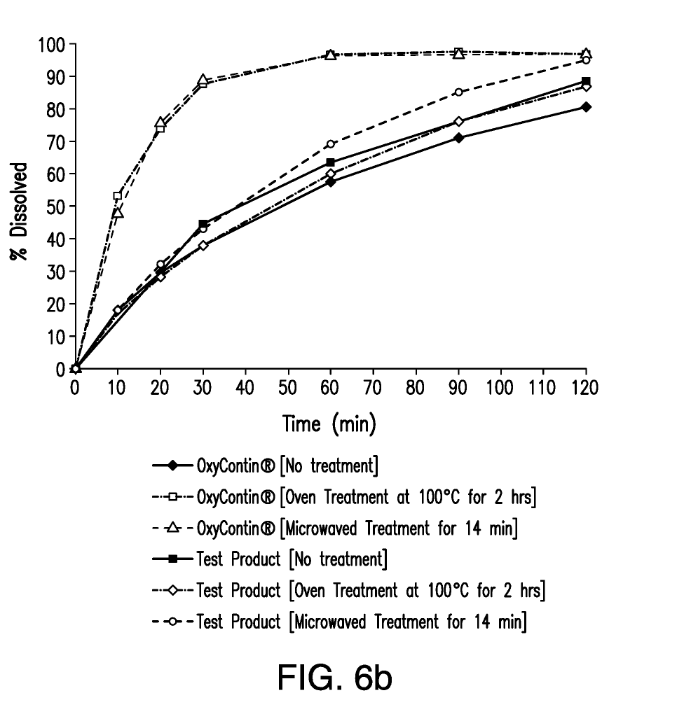

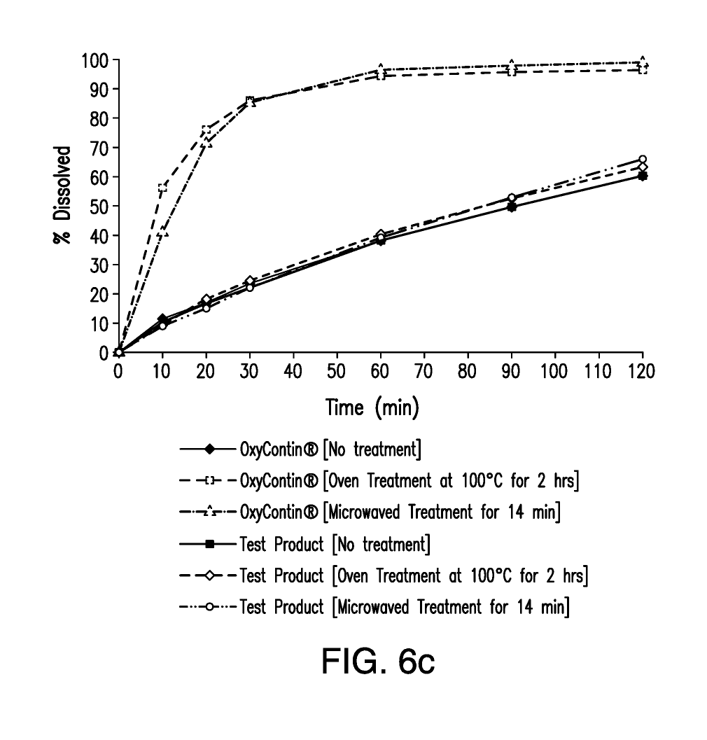

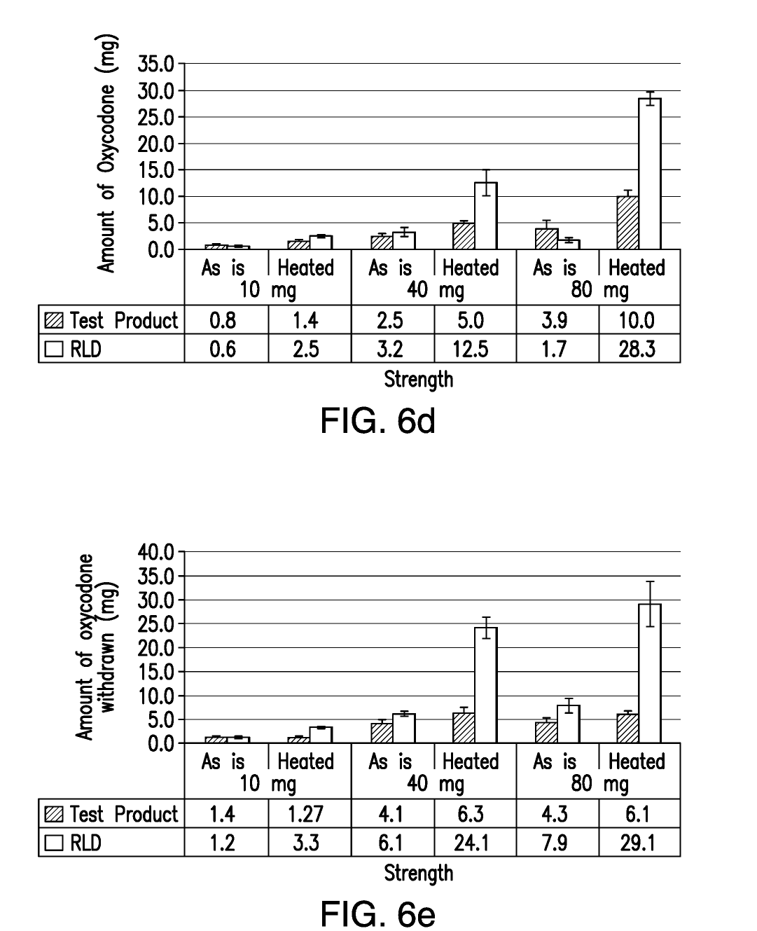

The oral tablets of the present technology (Test) were tested for extraction of oxycodone as follows. One tablet (cut into 4 pieces) was placed in a 300 mL Erlenmeyer flask; solvent (200 ml) was added and the solution was stirred for up to 22 hours. Intermittently, aliquots were removed and assayed for oxycodone content by HPLC. Results from these extraction studies (upon extraction for five hours with different solvents) are shown in FIG. 6a, demonstrating that the extraction of oxycodone is comparable to OXYCONTIN® 40 mg ORT tablets (RLD).

FIG. 6b demonstrates the results from extraction testing of oxycodone extended release tablets (cut into 4 pieces) according to the present technology (Test) with and without oven or microwave heat pretreatment compared to OXYCONTIN® 40 mg ORT tablets (after cutting into 4 pieces) in 500 mL water using dissolution USP Apparatus II (paddles at 150 rpm) at 37° C.

As seen in FIG. 6b, OXYCONTIN® ORT tablets begin to lose their extraction protection property (i.e., lose their resistance to extraction of active from the cut tablet) in water after oven heat pretreatment at 100° C. for 2 hours as well as after microwave heat pretreatment at 1200 W for 14 minutes. In contrast, oxycodone extended release tablets according to the present technology (Test) maintained their extraction protection in water after oven heat pretreatment at 100° C. for 2 hours as well as after microwaving at 1200 W for 14 minutes.

FIG. 6c demonstrates the results from extraction testing of oxycodone extended release tablets (cut into 4 pieces) according to the present technology (Test) in water:ethanol (1:1) after microwave or oven heat pretreatment compared to OXYCONTIN® 40 mg ORT tablets (after cutting into 4 pieces) in 500 mL water:ethanol (1:1) using dissolution USP Apparatus II (paddles at 150 rpm) at 37° C.

As seen in FIG. 6c, OXYCONTIN® ORT tablets begin to lose their extraction protection property in water:ethanol (1:1) after oven heat pretreatment at 100° C. for 2 hours as well as after microwave heat pretreatment at 1200 W for 14 minutes. In contrast, oxycodone extended release tablets according to the present technology maintained their extraction protection in water:ethanol (1:1) after each of the heat pretreatments.

FIGS. 6d and 6e show the amount of oxycodone hydrochloride withdrawn at 10 minutes, from the aqueous solution made at room temperature and at 90° C., respectively, of Test and OXYCONTIN® ORT tablets (RLD), with and without heat pretreatment, into a syringe with 27 gauge needle.

As seen in FIGS. 6d and 6e, the amount of oxycodone hydrochloride extracted from the heat pretreatment (“heated”) Test Product into aqueous solution and withdrawn into a 27 G needle, is less compared to the amount of oxycodone hydrochloride extracted from heat pretreatment OXYCONTIN® ORT tablets (RLD).

Polyox Molecular Weight Evaluation

Samples of oxycodone hydrochloride extended release tablets (Test), 40 mg, and OXYCONTIN® ORT tablets, 40 mg (RLD) were evaluated for PEO molecular weight after heat pretreatment.

Oven Treatment—For oxycodone hydrochloride tablets of the present technology (Test) and OXYCONTIN® ORT tablets (RLD), two tablets of each product were transferred into scintillation vials. The vials were put into an oven preheated to 100° C. The tablets were incubated at 100° C. for 2 hours.

Microwave Treatment—A microwave was preheated at 100% power until the temperature inside the microwave reached 80° C. Each tablet (e.g., Test and RLD) was placed (one at a time) on the center of the glass tray inside the microwave, and microwaved for 14 minutes at 1200 W (100% power).

The tablets used to prepare the sample solutions were without treatment, or heat pretreated (by oven or microwave). For each sample, two tablets were cut into small pieces (8 pieces) and transferred into a 100 mL volumetric flask. Water was added to the flask until two-thirds full. The sample was stirred at room temperature overnight. Water was added to bring the volume to 100 ml and mixed well. The solution was injected into the HPLC for analysis.

Analysis: The samples were analyzed with a Size Exclusion Column (Ultrahydrogel 2000) using a Charged Aerosol Detector. The analysis of the molecular weight of PEO was determined by size exclusion chromatography. The molecular weight of the polymer is reflected by the retention time of the peak (x-axis) (i.e., the longer the retention time, the smaller the molecular weight).

In the overlay of chromatograms of tablets of the present technology, the peak retention times of oxycodone tablets, whether not treated, heat pretreated by oven, or heat pretreated by microwave, did not show observed changes. This indicated that the molecular weights of PEO of these samples are similar and all comparable with the mixture of POLYOX™ standard with molecular weight 900,000 Da and 7,000,000 Da in a ratio of 1:1. PEO is prone to oxidation upon heat treatment with consequent breakdown in molecular weight (MW) of the polymer. The MW breakdown with formation of lower MW species can negatively impact the abuse-deterrent properties of the product.

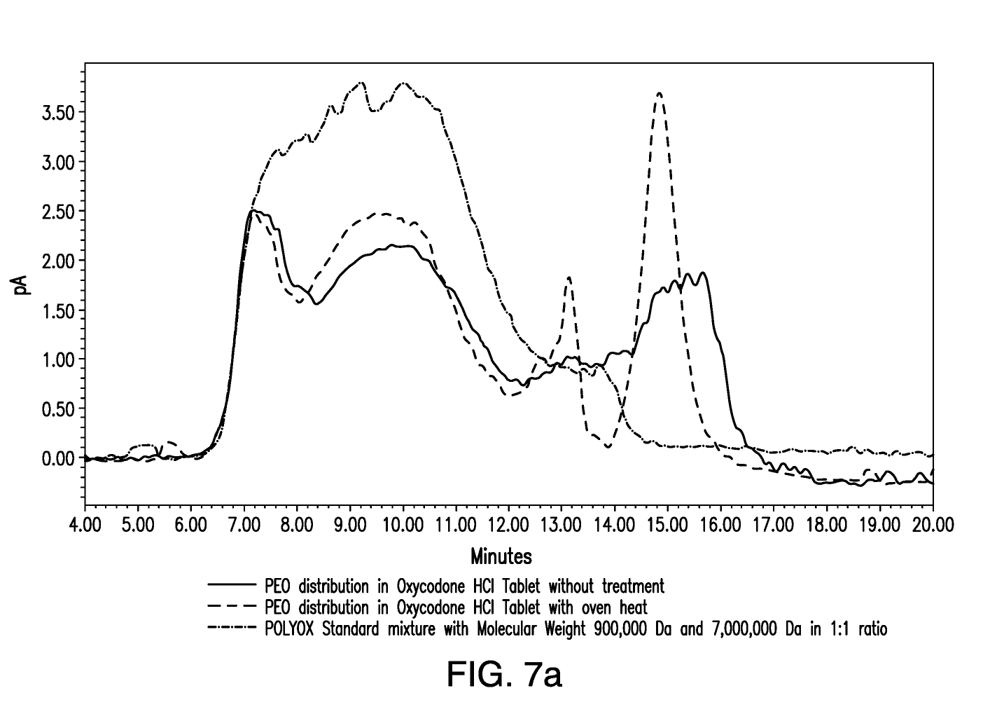

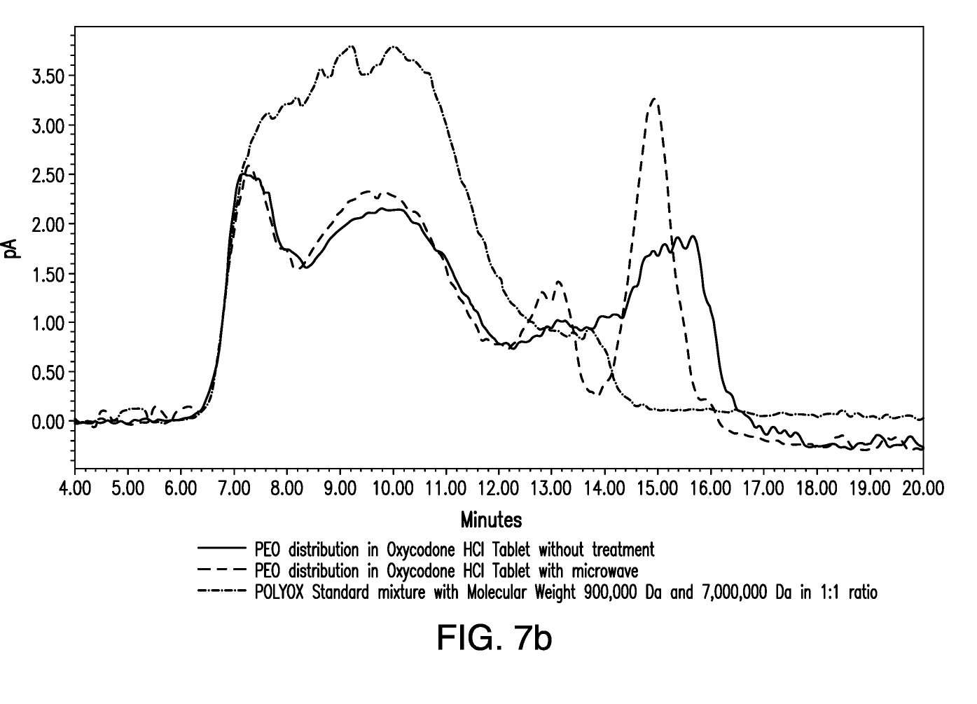

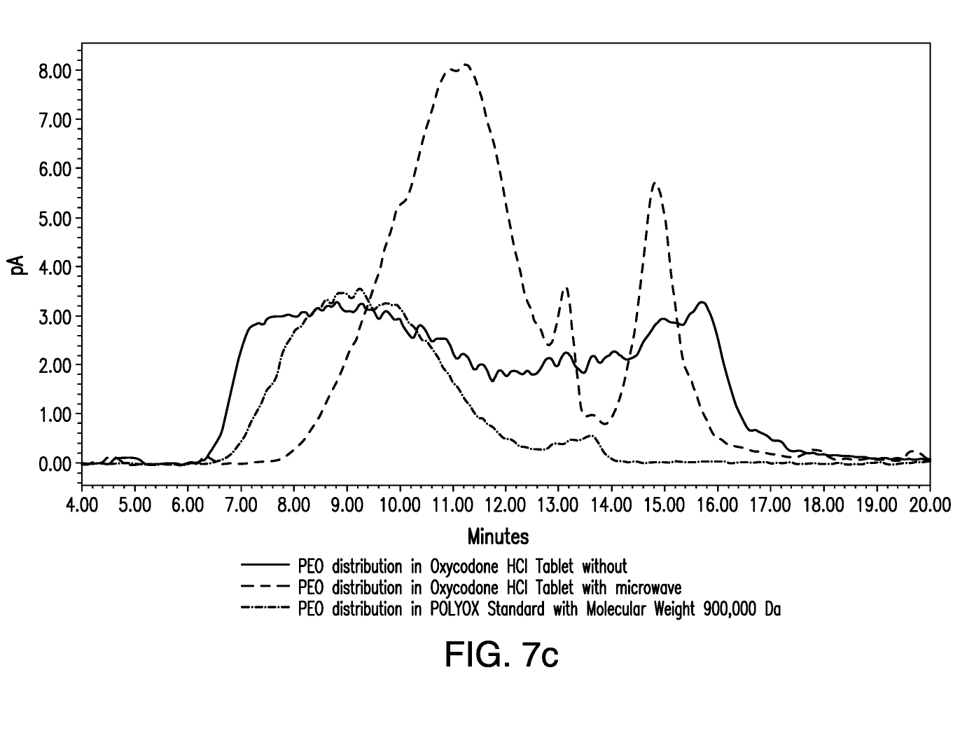

FIGS. 7a through 7c represent size exclusion chromatograms of PEO for OXYCONTIN® ORT tablets vs. Test (of the present technology) before and after heat treatments (e.g., heat pretreatments). The data on the x-axis represents retention time of PEO with different MW species; higher MW will have shorter retention times than lower MW species. The data on y-axis represents amount or concentration of PEO for different MW species. The data demonstrate that PEO in OXYCONTIN® ORT tablets breaks down after heat treatment, resulting in increases in lower MW species (and their concentrations) compared to without heat treatment (FIG. 7c). In contrast, such changes in MW and concentration of PEO observed upon heat treatment in the tablets of the present technology were considered insignificant (FIGS. 7a and 7b). The data show that the formulation of the present technology provides greater stability of the product, thereby maintaining its abuse-deterrent properties during heat treatment (e.g., heat pretreatment).

On the other hand, the molecular weight of PEO in oven treated OXYCONTIN® ORT tablets and microwave treated OXYCONTIN® tablets showed significant changes, and the retention times of the heat treated PEO are significantly longer than the nontreated sample. This indicates that the PEO molecular weight decreased dramatically compared to the nontreated sample. The molecular weight of the PEO in heat treated OXYCONTIN® ORT tablets is significantly smaller than 900,000 Da.

1-112. (canceled)

113. An extended release oral tablet composition comprising a matrix comprising a first component and a second component,
wherein the first component comprises a therapeutically effective amount of oxycodone hydrochloride in combination with a polyethylene oxide (PEO) polymer having a molecular weight of about 900,000 Dalton (Da) and an additional nonionic polymer;
wherein the second component comprises a PEO polymer having a molecular weight of about 900,000 Da and a PEO polymer having a molecular weight of about 7M Da;
wherein the first component is hot melt extruded or melt granulated, milled, and blended with the second component, and the blend is compressed into a tablet, and the tablet is cured for a period of at least 10 hours, and
wherein the composition exhibits enhanced heat stability with a deviation of no more than 5% increase in an in vitro dissolution rate in simulated gastric fluid (SGF), with or without 40% ethanol v/v, after heat pretreatment, when compared with a corresponding in vitro dissolution rate in SGF, with or without 40% ethanol v/v, without heat pretreatment.

114. The tablet composition of claim 113, wherein the total combined weight of the PEO polymers having molecular weights of about 900,000 Da and about 7M Da is less than about 65% by weight, based on the total weight of the composition, and the total weight of the PEO polymer having a molecular weight of about 7M Da is less than about 35% by weight, based on the total weight of the composition.

115. The tablet composition of claim 113, wherein the composition further comprises an antioxidant selected from the group consisting of d-alpha-tocopherol, polyethylene glycol 1000 succinate, ascorbic acid, dl-alpha-tocopherol, dl-alpha-tocopherol acetate, alpha-tocopherol, vitamin E, sodium citrate, and citric acid.

116. The tablet composition of claim 115, wherein the antioxidant is dl-alpha-tocopherol.

117. The tablet composition of claim 113, wherein the heat pretreatment comprises heating the composition in an oven at about 100° C. for about two hours, or heating the composition in a microwave, preheated to about 80° C., at about 1200 W for about 14 minutes.

118. The tablet composition of claim 113, wherein the in vitro dissolution rate of the composition is characterized by percentage amount of oxycodone hydrochloride released at two hours of dissolution in SGF, with or without 40% ethanol v/v, at 37° C.

119. The tablet composition of claim 113, wherein the tablet composition is cured for a period of between 14 and 18 hours.

120. An extended release oral tablet composition comprising a matrix comprising a first component and a second component,
wherein the first component comprises a therapeutically effective amount of oxycodone hydrochloride in combination with a PEO polymer having a molecular weight of about 900,000 Da, and an additional nonionic polymer;
wherein the second component comprises a PEO polymer having a molecular weight of about 900,000 Da and a PEO polymer having a molecular weight of about 7M Da;
wherein the first component is hot melt extruded or melt granulated, milled, and blended with the second component, and the blend is compressed into a tablet, and the tablet is cured for a period of at least 10 hours, and
wherein the composition exhibits enhanced heat stability, and resistance to drug segregation upon grinding measured as a variance of API segregation of less than 4 between a fines fraction with a particle size of less than about 75 microns and a coarse fraction with a particle size of between about 125 microns and about 250 microns.

121. The tablet composition of claim 120, wherein the tablet has a variance of API segregation of less than 3.

122. The tablet composition of claim 120, wherein the composition maintains an extended release profile of oxycodone hydrochloride, characterized by about 40% to about 44% release in two hours, after being subjected to heat pretreatment in an oven at a temperature of about 100° C. for about two hours, or in a microwave at 1200 W for about 14 minutes, and with a deviation of no more than about 5% from an extended release oxycodone hydrochloride oral tablet composition that is not subjected to heat pretreatment.

123. The tablet composition of claim 120, wherein an in vitro dissolution rate of the composition, characterized by the percent amount of oxycodone hydrochloride released at 90 minutes of dissolution in SGF comprising 40% ethanol v/v at 37° C., after being subjected to heat pretreatment in an oven at a temperature of about 100° C. for about two hours, deviates no more than about 5% from a corresponding in vitro dissolution rate of an extended release oxycodone hydrochloride oral tablet composition that is not subjected to heat pretreatment.

124. The tablet composition of claim 120, wherein the in vitro dissolution rate of the composition, characterized by the percent amount of oxycodone hydrochloride released at 90 minutes of dissolution in SGF comprising 40% ethanol v/v at 37° C., after being subjected to heat pretreatment in an oven at a temperature of about 100° C. for about two hours, deviates no more than about 5% from a corresponding in vitro dissolution rate of an extended release oxycodone hydrochloride oral tablet composition measured in SGF without ethanol.

125. The tablet composition of claim 120, wherein the in vitro dissolution rate of the composition, characterized by the percent amount of oxycodone hydrochloride released at 90 minutes of dissolution in SGF comprising 40% ethanol v/v at 37° C., deviates no more than about 5% from a corresponding in vitro dissolution rate, measured in SGF comprising 40% ethanol v/v at 37° C., of an extended release oxycodone hydrochloride oral tablet composition that is not made by a combination of (1) hot melt extrusion or melt granulation and (2) curing for a period of at least 10 hours.

126. The tablet composition of claim 120, wherein the in vitro dissolution rate of the composition, comprising the tablet cut into four pieces, characterized by the percent amount of oxycodone hydrochloride released at 90 minutes of dissolution in SGF comprising 40% ethanol v/v at 37° C., after being subjected to heat pretreatment in an oven at a temperature of about 100° C. for about two hours, deviates no more than about 5% from a corresponding in vitro dissolution rate of an extended release oxycodone hydrochloride oral tablet composition that is not subjected to cutting and heat pretreatment.

127. The tablet composition of claim 120, wherein oxycodone hydrochloride is fully embedded in a polymer matrix comprising PEO, hydroxypropyl methylcellulose, and a mixture of polyvinyl acetate, polyvinyl pyrrolidone, sodium lauryl sulfate, and silica.

128. The tablet composition of claim 120, wherein the resistance to drug segregation results in percentage of oxycodone hydrochloride in each of the fines fraction and the coarse fraction being in the range of about 90% to about 110% of that predicted from the composition of the tablet.

129. The tablet composition of claim 128, wherein the resistance to drug segregation of oxycodone hydrochloride in the fines fraction upon grinding results in a reduced amount of oxycodone hydrochloride available for insufflation.

130. A method of deterring abuse of oxycodone hydrochloride, the method comprising providing an extended release oral tablet composition to a subject in need thereof,
wherein the composition comprises a matrix comprising a first component and a second component;
wherein the first component comprises a therapeutically effective amount of oxycodone hydrochloride in combination with a PEO polymer having a molecular weight of about 900,000 Da, and an additional nonionic polymer;
wherein the second component comprises a PEO polymer having a molecular weight of about 900,000 Da and a PEO polymer having a molecular weight of about 7M Da; and
wherein the first component is hot melt extruded or melt granulated, milled, and blended with the second component, and the blend is compressed into a tablet, and the tablet is cured for a period of at least 10 hours.

131. The method of claim 130, wherein the composition provides an extended release of oxycodone hydrochloride and exhibits enhanced heat stability and higher resistance to drug segregation compared with an extended release oxycodone hydrochloride oral tablet composition that is not made by a combination of (1) hot melt extrusion or melt granulation and (2) curing for a period of at least 10 hours.

132. A process for making an extended release, tamper resistant, abuse-deterrent oral tablet composition, comprising:
mixing a therapeutically effective amount of oxycodone hydrochloride, at least one PEO polymer having a molecular weight of about 900,000 Da, and at least one rate limiting nonionic polymer, and hot melt extruding or melt granulating the mixture to form a first component;
milling the first component;
mixing a PEO polymer having a molecular weight of about 900,000 Da, a PEO polymer having a molecular weight of about 7M Da, and a stabilizing agent to form a second component;
blending the first component and the second component into a uniform blend;
compressing the uniform blend to form a tablet; and
curing the tablet for at least 10 hours.

133. The process of claim 132, wherein the stabilizing agent is dl-alpha-tocopherol.

134. An extended release oral tablet composition comprising a matrix comprising a first component and a second component,
wherein the first component comprises a therapeutically effective amount of oxycodone hydrochloride in combination with a PEO polymer having a molecular weight of about 900,000 Da, and an additional nonionic polymer;
wherein the second component comprises a PEO polymer having a molecular weight of about 900,000 Da and a PEO polymer having a molecular weight of about 7M Da;
wherein the first component is hot melt extruded or melt granulated, milled, and blended with the second component, and the blend is compressed into a tablet, and the tablet is cured for a period of at least 10 hours; and
wherein the composition is formulated to provide resistance to syringeability by limiting the extractability of oxycodone hydrochloride such that less than about 20% of the oxycodone hydrochloride is available in syringeable form.

135. The tablet composition of claim 134, wherein less than about 15% of the oxycodone hydrochloride is available in syringeable form.

136. The tablet composition of claim 135, wherein less than about 10% of the oxycodone hydrochloride is available in syringeable form.

137. The tablet composition of claim 134, wherein the syringeable form is a syringeable liquid obtained by adding at least one crushed or ground tablet to 10 ml of water at room temperature, forming a suspension, vortexing the suspension, and maintaining the suspension for about 10 minutes.

138. The tablet composition of claim 134, wherein the syringeable form is a syringeable liquid obtained by adding at least one crushed or ground tablet to 10 ml of water at 90° C., forming a suspension, vortexing the suspension, and maintaining the suspension for about 10 minutes.

139. The tablet composition of claim 137, wherein the syringeable liquid is withdrawn through an 18-gauge needle into a 10 ml syringe.

140. The tablet composition of claim 137, wherein the dosage form is crushed or ground after heat pretreatment in an oven at a temperature of about 100° C. for about two hours.

141. The tablet composition of claim 137, wherein the dosage form is crushed or ground after heat pretreatment in a microwave at about 1200 W for about 14 minutes.
